(19) United States
(12) Patent Application Publication
(10) Pub. No.: US 2026/0076969 A1
(43) Pub. Date: Mar. 19, 2026

(54) METHODS AND COMPOSITIONS FOR THE TREATMENT OF CANCER

(71) Applicant: Board of Regents, The University of Texas System, Austin, TX (US)

(72) Inventors: Yonathan Lissanu, Houston, TX (US); Nicholas Blazanin, Houston, TX (US)

(73) Assignee: Board of Regents, The University of Texas System, Austin, TX (US)

(21) Appl. No.: 19/323,888

(22) Filed: Sep. 9, 2025

Related U.S. Application Data
(60) Provisional application No. 63/694,551, filed on Sep. 13, 2024.

(51) Int. Cl. A61K 31/517; A61K 31/155; A61K 31/40; A61K 31/454; A61K 45/06; A61P 35/00

(52) U.S. Cl. CPC A61K 31/517; A61K 31/155; A61K 31/40; A61K 31/454; A61K 45/06; A61P 35/00

(57) ABSTRACT

The present disclosure provides compositions and methods for the treatment of cancer. The present disclosure further provides therapeutic and pharmaceutical compositions comprising a ROCK inhibitor and an OXPHOS inhibitor. Aspects of the disclosure further relate to methods for treating diseases or disorders associated with a mutation in a subunit of a SWI/SNF chromatin remodeling complex.

### CROSS-REFERENCE TO RELATED APPLICATIONS

This application claims the priority of U.S. Provisional Appl. Ser. No. 63/694,551, filed Sep. 13, 2024, the entire disclosure of which is incorporated herein by reference.

### STATEMENT OF GOVERNMENT RIGHTS

This invention was made with government support under CA251629 awarded by the National Institutes of Health. The government has certain rights in the invention.

### FIELD OF THE INVENTION

This present disclosure relates to the field of cancer therapeutics, and more specifically to compositions and methods for targeting the metabolic dysregulation of cancer.

### BACKGROUND OF THE INVENTION

Metabolic deregulation is one of the hallmarks of cancer. Despite intense efforts, therapeutic strategies targeting metabolic pathways have been unsuccessful. This is primarily due to a rapid adaptive response by cancer cells in response to the inhibition of key metabolic pathways. It is, therefore, unlikely that single agent therapies targeting a metabolic pathway will be efficacious. Thus, new combination treatment regimens need to be developed. The present disclosure provides such novel and effective therapeutic regimens. In particular, the present disclosure demonstrates that the combination of inhibitors of oxidative phosphorylation (OXPHOS) and inhibitors of Rho associated protein kinase (ROCK) have synergistic anti-cancer activity. In particular, the combination of OXPHOS and ROCK inhibition induced profound tumor growth inhibition at doses where single agent therapy showed no tumor growth effect. The present disclosure demonstrates that this phenomenon is due to complementary elimination of adaptive responses generated by cancer cells in response to each individual agent.

### SUMMARY OF THE INVENTION

In one aspect, the present disclosure provides a method of treating a subject afflicted with or at risk of developing a cancer comprising a mutation in a subunit of a SWI/SNF chromatin remodeling complex, the method comprising administering to the subject an effective amount of a ROCK inhibitor and an effective amount of an OXPHOS inhibitor. In one embodiment, the ROCK inhibitor is selected from the group consisting of belumosudil (KD025), AT-13148, BA-210, β-elemene, chroman 1, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, and Y-39983. In another embodiment, the OXPHOS inhibitor is selected from the group consisting of IACS-010759 (IACS-10759), metformin, phenformin, HP661, IM156 (lixumistat, HL156A), BAY 87-2243, VLX600, lonidamine, atovaquone, AG311, Mito-Met₁₀ (norMitoMet), mubritinib, carboxyamidotriazole (CAI), ME344, fenofibrate, deguelin, papaverine, α-TOS, neoantimycin F, ADDA 5, Gboxin, S-Gboxin, oligomycin A, apoptolidin, bedaquiline, DX3-213B, BAY-179, 4-methyl-2-oxovaleric acid (ketoleucine, 4-MOV, KIC), diphenylamine hydrochloride, and carbonylcyanide 3-chlorophenylhydrazone (CCCP), carbonyl cyanide 4-(trifluoromethoxy)phenylhydrazone (FCCP), 3-nitropropionic acid, amobarbital, antimycin A, arsenic trioxide, atpenin A5, aurovertin B, BAM 15, Bz-423, berberine, canagliflozin, calcimycin (A-23187), cyanine5 alkyne (alkyne-Cy5), DX2-201, DX3-234, DX3-235, hydrocortisone, IM176OUT05, malonate, mIBG, Mito-LND (Mito-Lonidamine), Mito-Q, MPTP (1-methyl 4-phenyl 1,2,3,6 tetrahydropyridine), myxothiazol nitric oxide, nefazodone, nonactin, parimifasor (LYC-30937), piericidin A, pioglitazone, pyrvinium, ranolazine, rosiglitazone, rotenone, RTB70, TRC1, OXPHOS-IN-1, SMV-32, stigmatellin, TRAP1-IN-2, TRAP1-IN-1, SCAL-255, SCAL-266, siccanin, tetrathiomolybdate, tiabendazole, and TTFA (thenoyltrifluoroacetone). In yet another embodiment, the ROCK1 inhibitor is belumosudil (KD025) and the OXPHOS inhibitor is IACS-010759 (IACS-10759), metformin, phenformin, or IM156. The subunit of the SWI/SNF chromatin remodeling complex, in still yet another embodiment, is ARID1A or SMARCA4. In one embodiment, the cancer resistant to chemotherapy, immunotherapy, or an inhibitor of KRAS.

The effective amount of the ROCK1 inhibitor, in another embodiment, is about 1 mg/kg to about 2500 mg/kg body weight, about 10 mg/kg to about 2000 mg/kg body weight, about 50 mg/kg to about 1750 mg/kg body weight, about 100 mg/kg to about 1500 mg/kg body weight, about 200 mg/kg to about 1200 mg/kg body weight, about 100 mg/kg to about 800 mg/kg body weight, about 100 mg/kg to about 600 mg/kg body weight, about 200 mg/kg boy weight to about 500 mg/kg body weight, or about 200 mg/kg to about 400 mg/kg body weight. The effective amount of the ROCK1 inhibitor, in yet another embodiment, is about 1 mg/kg to about 2500 mg/kg body weight per day, about 10 mg/kg to about 2000 mg/kg body weight per day, about 50 mg/kg to about 1750 mg/kg body weight per day, about 100 mg/kg to about 1500 mg/kg body weight per day, about 200 mg/kg to about 1200 mg/kg body weight per day, about 100 mg/kg to about 800 mg/kg body weight per day, about 100 mg/kg to about 600 mg/kg body weight per day, about 200 mg/kg boy weight to about 500 mg/kg body weight per day, or about 200 mg/kg to about 400 mg/kg body weight per day. The effective amount of the OXPHOS inhibitor, in still yet another embodiment, is about 0.5 mg/kg to about 2500 mg/kg body weight, about 10 mg/kg to about 2000 mg/kg body weight, about 50 mg/kg to about 1750 mg/kg body weight, about 200 mg/kg to about 1000 mg/kg body weight, about 200 mg/kg to about 800 mg/kg body weight, about 400 mg/kg to about 600 mg/kg body weight, about 0.5 mg/kg to about 20 mg/kg body weight, about 0.5 mg/kg to about 15 mg/kg body weight, about 0.5 mg/kg to about 10 mg/kg body weight, about 0.5 mg/kg to about 9 mg/kg body weight, about 0.5 mg/kg to about 8 mg/kg body weight, about 0.5 mg/kg to about 7 mg/kg body weight, about 0.5 mg/kg to about 6 mg/kg body weight, about 0.5 mg/kg to about 5 mg/kg body weight, about 0.5 mg/kg to about 4 mg/kg body weight, about 0.5 mg/kg to about 3 mg/kg body weight, about 0.5 mg/kg to about 2 mg/kg body weight, or about 0.5 mg/kg to about 1.5 mg/kg body weight. The effective amount of the OXPHOS inhibitor, in one embodiment, is about 0.5 mg/kg to about 2500 mg/kg body weight per day, about 10 mg/kg to about 2000 mg/kg body weight per day, about 50 mg/kg to about 1750 mg/kg body weight per day, about 200 mg/kg to about 1000 mg/kg body weight per day, about 200 mg/kg to about 800 mg/kg body weight per day, about 400 mg/kg to about 600 mg/kg body weight per day, about 0.5 mg/kg to about 20 mg/kg body weight per day, about 0.5 mg/kg to about 15 mg/kg body weight per day, about 0.5 mg/kg to about 10 mg/kg body weight per day, about 0.5 mg/kg to about 9 mg/kg body weight per day, about 0.5 mg/kg to about 8 mg/kg body weight per day, about 0.5 mg/kg to about 7 mg/kg body weight per day, about 0.5 mg/kg to about 6 mg/kg body weight per day, about 0.5 mg/kg to about 5 mg/kg body weight per day, about 0.5 mg/kg to about 4 mg/kg body weight per day, about 0.5 mg/kg to about 3 mg/kg body weight per day, about 0.5 mg/kg to about 2 mg/kg body weight per day, or about 0.5 mg/kg to about 1.5 mg/kg body weight per day.

In one embodiment, the cancer is selected from the group consisting of lung cancer, non-small cell lung cancer (NSCLC), small cell lung cancer (SCLC), brain cancer, glioblastoma, medulloblastoma, skin cancer, melanoma, pancreatic cancer, colorectal cancer, appendiceal cancer, hematopoietic cancer, B-cell lymphoma, leukemia, myeloma, breast cancer, head and neck cancer, prostate cancer, kidney cancer, bladder cancer, liver cancer, esophageal cancer, stomach cancer, thyroid cancer, small bowel adenocarcinoma, hepatobiliary cancer, gynecological cancer, cervical cancer, uterine cancer, and ovarian cancer. In another embodiment, the subject is a mammalian subject. In yet another embodiment, the subject is a human subject. The administering, in still yet another embodiment, comprises oral administration, buccal administration, injection, microneedle administration, vaginal administration, inhalation, intraosseous administration, transnasal application, topical administration, transdermal application, or rectal administration. In one embodiment, the methods of the present disclosure may further comprise administering a second therapy to the subject. The second therapy, in another embodiment, may be selected from the group consisting of chemotherapy, radiation therapy, immunotherapy, and surgery.

In certain embodiments, the methods of the present disclosure may comprise administering a pharmaceutical composition comprising the effective amount of the ROCK inhibitor or the effective amount of the OXPHOS inhibitor to the subject. In one embodiment, the methods of the present disclosure may comprise administering a first pharmaceutical composition comprising the effective amount of the ROCK inhibitor and a second pharmaceutical composition comprising the effective amount of the OXPHOS inhibitor to the subject. In another embodiment, the pharmaceutical composition comprises the effective amount of the ROCK inhibitor and the effective amount of the OXPHOS inhibitor.

In another aspect, the present disclosure provides a pharmaceutical composition comprising an effective amount of a ROCK inhibitor and an effective amount of an OXPHOS inhibitor. Non-limiting examples of ROCK inhibitors include belumosudil (KD025), AT-13148, BA-210, β-elemene, chroman 1, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, and Y-39983. Non-limiting examples of OXPHOS inhibitors include IACS-010759 (IACS-10759), metformin, phenformin, HP661, IM156 (lixumistat, HL156A), BAY 87-2243, VLX600, lonidamine, atovaquone, AG311, Mito-Met₁₀ (norMitoMet), mubritinib, carboxyamidotriazole (CAI), ME344, fenofibrate, deguelin, papaverine, α-TOS, neoantimycin F, ADDA 5, Gboxin, S-Gboxin, oligomycin A, apoptolidin, bedaquiline, DX3-213B, BAY-179, 4-methyl-2-oxovaleric acid (ketoleucine, 4-MOV, KIC), diphenylamine hydrochloride, and carbonylcyanide 3-chlorophenylhydrazone (CCCP), carbonyl cyanide 4-(trifluoromethoxy)phenylhydrazone (FCCP), 3-nitropropionic acid, amobarbital, antimycin A, arsenic trioxide, atpenin A5, aurovertin B, BAM 15, Bz-423, berberine, canagliflozin, calcimycin (A-23187), cyanine5 alkyne (alkyne-Cy5), DX2-201, DX3-234, DX3-235, hydrocortisone, IM176OUT05, malonate, mIBG, Mito-LND (Mito-Lonidamine), Mito-Q, MPTP (1-methyl 4-phenyl 1,2,3,6 tetrahydropyridine), myxothiazol nitric oxide, nefazodone, nonactin, parimifasor (LYC-30937), piericidin A, pioglitazone, pyrvinium, ranolazine, rosiglitazone, rotenone, RTB70, TRC1, OXPHOS-IN-1, SMV-32, stigmatellin, TRAP1-IN-2, TRAP1-IN-1, SCAL-255, SCAL-266, siccanin, tetrathiomolybdate, tiabendazole, and TTFA (thenoyltrifluoroacetone). In one embodiment, the ROCK1 inhibitor is belumosudil (KD025) and the OXPHOS inhibitor is IACS-010759 (IACS-10759), metformin, phenformin, or IM156. In another embodiment, the pharmaceutical composition is formulated for oral administration, buccal administration, injection, microneedle administration, vaginal administration, inhalation, intraosseous administration, trans nasal application, topical administration, transdermal application, or rectal administration. In yet another embodiment, the pharmaceutical composition is serum-free, endotoxin-free, or sterile.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 demonstrates that CRISPR and clinical drug screening identified ROCK1/2 as a synergistic combinatorial agent with OXPHOS inhibition in SMARCA4-mutant lung cancer. FIG. 1, Panel A—Schematic representation of the workflow for CRISPR screens performed in A549, H1299, and H2023 SMARCA4-mutant lung cancer cells. FIG. 1, Panel B—NormZ scores of candidate genes common at both early and late timepoints from the FDAome library CRISPR screening results in H1299 cells cultured in presence or absence of 4 nM IACS-10759. The NormZ score was used to define a possible synthetic lethal interaction with IACS-10759. All genes targeted by the FDAome library were scored according to the fold change of levels of their respective sgRNAs. Genes whose loss of function led to IACS-10759 sensitivity appear on the bottom left quadrant, and genes whose loss of function led to IACS-10759 resistance appear on the top right quadrant. High-confidence candidate genes are shown in dark grey and those selected for further analysis are indicated in black. FIG. 1, Panel C—List of selected therapeutics and respective gene targets that are FDA approved or in clinical development tested in combination with IACS-10759 for synergistic effects on cell growth. FIG. 1, Panel D—Overall synergy scores determined in A549, H1299, and H2023 cells exposed to therapeutics against selected CRISPR hits in combination with IACS-10759. An overall synergy score>10 indicates the interaction between two drugs is likely to be synergistic. Each combination was performed in triplicate for each cell line. FIG. 1, Panel E—Dose-response curves of H1299 cells exposed to increasing concentrations of KD025 in the presence or absence of IACS-10759. Cell growth (measured as % confluency) was assayed 5 days after drug exposure. Data are presented as mean+/−SEM of three independent experiments. FIG. 1, Panel F—Clonogenic growth assays of H1299 cells cultured in the presence of KD025 and IACS-10759 alone or in combination for 12 days. Surviving cells after the treatment were fixed and visualized by crystal violet staining. Representative images of three independent experiments are shown. FIG. 1, Panel G—Dose-response curves of H1299 cells exposed to increasing concentrations of KD025 in the presence or absence of IM156. Cell growth (measured as % confluency) was assayed 5 days after drug exposure. Data are presented as mean+/−SEM of three independent experiments. FIG. 1, Panel F—Clonogenic growth assays of H1299 cells cultured in the presence of KD025 and IM156 alone or in combination for 12 days. Surviving cells after the treatment were fixed and visualized by crystal violet staining. Representative images of three independent experiments are shown. FIG. 1, Panel I—Overall synergy scores determined in H1299 cells exposed to the indicated OXPHOS inhibitors in combination with KD025. An overall synergy score>10 indicates the interaction between two drugs is likely to be synergistic. Each combination was performed in triplicate for each cell line.

FIG. 2 demonstrates that ROCK blockade in combination with OXPHOS inhibition induces cell cycle growth arrest, cell death, and severe energy stress. FIG. 2, Panel A—FUCCI cell cycle analysis showing the percentage of H1299 cells in M-G1, G1, G1/S, or late S/G2/M following treatment with DMSO, IACS-10759, KD025, or the combination over 96 hr. Percentage of cells at each cell cycle phase were quantitatively assessed by cell-by-cell analysis software from Incucyte S3. Data indicate mean+/−SEM from n=3 independent experiments. FIG. 2, Panel B and Panel C—Quantitation of apoptosis using either Cytotox green or Annexin V showing the percentage of apoptotic cells over 96 hr (Panel B) or at increasing doses of KD025 after 96 hr (Panel C), as assessed by cell Incucyte analysis following treatment with DMSO, IACS-10759, KD025, or the combination in H1299 cells. FIG. 2, Panel D—Seahorse mitochondrial stress test assay showing a representative trace measuring the mitochondrial oxygen consumption rate (OCR) in H1299 cells cultured with DMSO, KD025, IACS-10759, or the combination for 6 hr from which basal respiration, maximum respiratory capacity, ATP-linked respiration, and spare capacity were calculated (FIG. 2, Panel E). Data indicate mean+/−SEM from DMSO, n=4, IACS-10759, n=7, KD025, n=11, and combination, n=7 independent experiments. One-way ANOVA was used corrected for multiple comparisons. ****p-values<0.0001, ***, **, *. FIG. 2, Panel F—Seahorse glycolysis stress test assay showing a representative trace measuring the extracellular acidification rate (ECAR) in H1299 cells cultured with DMSO, KD025, IACS-10759, or the combination for 6 hr from which glycolysis, glycolytic capacity, and glycolytic reserve were calculated (FIG. 2, Panel G). Data indicate mean±SEM from DMSO, n=10, IACS-10759, n=11, KD025, n=10, and combination, n=9 independent experiments. One-way ANOVA was used corrected for multiple comparisons. ****p-values<0.0001, ***, **, *, ns, not significant. FIG. 2, Panel H, Panel J, and Panel I—Quantitation of ATP production by Seahorse XF real-time ATP rate assay following treatment with DMSO, IACS-10759, KD025, or the combination for 6 hr in H1299 cells from which total ATP production (Panel H), mitochondrial ATP production (Panel I), and glycolytic ATP production (Panel J) rates were calculated. Data shown are mean±SEM from DMSO, n=10, IACS-10759, n=11, KD025, n=10, and combination, n=9 independent experiments. One-way ANOVA was used corrected for multiple comparisons. ****p-values<0.0001, ***, **, *, ns, not significant. FIG. 2, Panel K—Bioenergetic profile map in H1299 cells produced by plotting basal OCR and ECAR of the indicated treatment groups.

FIG. 3 demonstrates that KD025 causes metabolic reprogramming by suppressing adaptive increase in glycolysis due to OXPHOS inhibition. FIG. 3, Panel A—Heatmap of the significantly different metabolite abundances involved in glycolysis, the pentose phosphate pathway, and the TCA cycle following treatment with DMSO, KD025, IACS-10759, or the combination for 24 and 48 hr in H1299 cells. Log 2 FC>0 represents an increase of metabolite abundance and Log 2 FC<0 represents a decrease of metabolite abundance. FIG. 3, Panel B—Relative abundance of select metabolites from glycolysis, the pentose phosphate pathway, and the TCA cycle pathway following treatment with DMSO, KD025, IACS-10759, or the combination for 24 and 48 hr. Metabolite abundance is expressed as relative peak intensity. Data indicate mean±SEM of n=3 independent experiments. FIG. 3, Panel C—Glucose uptake in H1299 cells following 6 hr of treatment as measured by uptake of 2-deoxyglucose and normalized to cell number. FIG. 3, Panel D—Lactate levels accumulated in the extra cellular media of H1299 cells treated for 6 hours were measured and normalized to the endpoint cell number. FIG. 3, Panel E—Heatmap of significantly labeled metabolites after isotope incorporation. Data reflect the relative sum of abundance of all ¹³C isotopologues. FIG. 3, Panel F, Panel G, Panel H, and Panel I—Fractional isotopic incorporation of ¹³C₆-glucose into glycolytic and TCA cycle metabolite intermediates as measured by GC/MS in H1299 cells following treatment with DMSO, KD025, IACS-10759, or the combination, and culture in ¹³C₆-glucose containing medium for 24 hr. m, number of labeled carbons. Data indicate mean±SEM of n=3 independent experiments.

FIG. 4 demonstrates the acute changes in phosphorylation and protein expression observed in H1299 lung cancer cells treated with DMSO, KD025, IACS-10759, and the combination by mass spectrometry-based proteomic and phosphoproteomic analyses. FIG. 4, Panel A—Volcano plot showing the impact of IACS-10759, KD025, or the combination on the global proteome. FIG. 4, Panel B—Volcano plot showing the impact of IACS-10759, KD025, or the combination on the global phosphoproteome. FIG. 4, Panel C—Venn diagram showing the overlap of phosphosites for IACS-10759, KD025, and the combination. FIG. 4, Panel D—Motif analysis of significantly upregulated and downregulated phosphosites in the IACS-10758/KD025 combination after 6 hr treatment.

FIG. 5 demonstrates the anti-tumor activity of the KD025 and IACS-10759 combination in vivo. FIG. 5, Panel A—In vivo H1299 xenograft model showing anti-tumor efficacy of IACS-10759 and KD025 alone or in combination after daily administration by oral gavage over 21 days. FIG. 5, Panel B—In vivo A549 xenograft model showing anti-tumor efficacy of IACS-10759 and KD025 alone or in combination after daily administration by oral gavage over 47 days. FIG. 5, Panel C—Comparison of final tumor volumes of H1299 tumor xenografts on day 21 (left panel) and of A549 tumor xenografts on day 47 (right panel). FIG. 5, Panel D—Body weight changes of each treatment group during the course of the experiment in H1299 (top) and A549 (bottom) tumor xenografts.

FIG. 6 shows the results of an in vivo study using a TC680 SMARCA4-mutant PDX model in NSG mice. IACS-10759 and KD025 (Belumosudil mesylate) were administered alone or in combination at the indicated doses by oral gavage 5×/week. FIG. 6, Panel A shows a schematic of the treatment schedule. FIG. 6, Panel B shows the individual volumes of each treatment group. TGI=Tumor Growth Inhibition. FIG. 6, Panel C shows the average tumor volume of each treatment group through duration of experiment.

FIG. 7 shows the results of an in vivo tumor study using a TC314 SMARCA4-mutant PDX model in NSG mice. IACS-10759 and KD025(Belumosudil mesylate) were administered alone or in combination at the indicated doses by oral gavage 5×/week. FIG. 7, Panel A shows a schematic of treatment schedule. FIG. 7, Panel B shows individual volumes of each treatment group. TGI=Tumor Growth Inhibition. FIG. 7, Panel C shows the average tumor volume of each treatment group through duration of experiment.

FIG. 8 demonstrates that ROCK blockade combined with OXPHOS inhibition causes cell cycle growth arrest and apoptosis following only a single dose. FIG. 8, Panel A shows cell cycle changes after single treatment of IACS-10759 and KD025 alone or in combination using indicated concentrations in H1299 cells. Cell cycle progression was assessed using a cell cycle reporter (FUCCI) and analyzed using Incucyte cell by cell analysis software. FIG. 8, Panel B shows analysis of cell death in H1299 cells after single treatment of IACS-10759 and KD025 alone or in combination using indicated concentrations. FIG. 8, Panel C shows analysis of cell death in H1299 cells after single treatment of IM156 and KD025 alone or in combination using indicated concentrations.

### DETAILED DESCRIPTION OF THE INVENTION

The present disclosure provides compositions and method for the treatment of a subject afflicted with or at risk of developing a cancer comprising a mutation in a subunit of a SWI/SNF chromatin remodeling complex. Genomic sequencing studies across numerous solid tumor types have demonstrated a high frequency of genetic alterations in multiple subunits of the SWI/SNF chromatin remodeling complex including SMARCA4 (also known as BRG1) and ARID1A ranging from 16% in early-stage disease to 33% in advanced lung cancer. Furthermore, a meta-analysis of 44 genomic studies has shown that 20% of all solid tumors have mutations in subunits of the SWI/SNF complex making it one of the most frequently mutated complexes in cancer. The SWI/SNF (SWItch/Sucrose Non-Fermenting) complex is a large multi-protein assembly that uses the energy derived from ATP hydrolysis to remodel nucleosomes and facilitate major chromatin dependent cellular processes such as DNA replication, repair, and transcription. There is intense effort to identify synthetic lethal or other vulnerabilities in SWI/SNF-mutant cancer. Most mutations in the SWI/SNF complex are inactivating and cannot be directly targeted therapeutically. In this regard, several vulnerabilities have been reported that enhance sensitivity of SMARCA4-mutant cancers such as inhibition of Aurora kinase A, CDK4/6, EZH2, ATR, and KDM6 methyltransferase. However, none of these have progressed into advanced clinical studies.

It has been challenging to develop efficacious small molecule inhibitors targeting OXPHOS with favorable safety and tolerability. One class of OXPHOS inhibitors include metformin and its related class of compounds. These drugs demonstrate a robust safety profile with limited toxicity, but display several limitations, including inadequate potency, “off target” toxicity, and undesirable in vivo pharmacokinetics. These limitations restrict the clinical use of metformin and related compounds as therapeutic agents for the treatment of cancer. IACS-10759 is highly selective and potent small-molecule inhibitor of complex I of the mitochondrial electron transport chain with favorable in vivo pharmacokinetic. While IACS-10759 showed promise in several preclinical cancer models, phase I clinical trials exposed dose-restricting toxicities (i.e., lactic acidosis; neurotoxicity) with only modest anti-tumor activity at tolerable doses.

Due to the current limitations of single agent OXPHOS inhibitor therapy, further clinical optimization through the discovery of potent and efficacious combination treatment regimens is required. In particular, no proposed combination treatment regimen exists for patients having SWI/SNF-mutant tumors. To identify such efficacious combination treatment regimens, the present disclosure describes CRISPR screens using a sgRNA library containing genes whose targets have therapeutics in advanced clinical development. By using doses of IACS-10759 that are clinically tolerated in three SMARCA4-mutant cell lines, several gene targets that are sensitive to low dose OXPHOS inhibition were identified. Validation of these targets through drug screening demonstrated the synergistic effect of combining OXPHOS and ROCK inhibition. Combination treatment with a ROCK inhibitor (KD025) and an OXPHOS inhibitor (IACS-10759) caused severe energetic stress by interrupting energy producing pathways and as a result had a profound impact on cell growth and survival.

### A. ROCK Inhibitors

ROCK1 and ROCK2 are serine/threonine kinases that regulate actin cytoskeletal rearrangements involved in cancer cell migration, motility, and invasion as well as glucose homeostasis. Inhibitors of ROCK may act through various mechanisms to reduce the activity or function of ROCK. In some embodiments, a ROCK inhibitor of the present disclosure may result in decreased cell migration, motility, and/or invasion. A ROCK inhibitor of the present disclosure, in particular embodiments, may reduce glycolytic capacity or glycolytic reserves in a cell. As used herein the term “ROCK inhibitor” refers to a compound that may bind to, decrease, prevent, delay activation, inactivate, desensitize, or downregulate the activity or expression of ROCK1 and/or ROCK2. Inhibitors of ROCK may also include, in certain embodiments, genetically modified versions of ROCK, e.g., versions with altered activity, as well as naturally occurring and synthetic antagonists, antibodies, peptides, cyclic peptides, nucleic acids, antisense molecules, ribozymes, small chemical molecules, and the like. Assays for inhibitors include, e.g., expressing ROCK1 and/or ROCK2 in vitro, in cells, or in cell membranes, applying putative inhibitor compounds, and then determining the functional effects on activity, as described herein.

Test samples or assays comprising ROCK that are treated with a potential inhibitor may be compared to a control sample lacking the inhibitor in order to determine the extent of inhibition. Control samples to which a test sample or assay is compared may be assigned a relative protein activity value of 100%. Inhibition of ROCK is achieved when the activity value of the test sample relative to the control sample is less than about 80%, including less than about 75%, about 70%, about 65%, about 60%, about 55%, about 50%, about 45%, about 40%, about 35%, about 30%, about 25%, about 20%, about 15%, about 10%, about 5%, and about 1%, including all ranges and values derivable therebetween.

Any ROCK inhibitor may be used according to the embodiments of the present disclosure. Non-limiting examples of such therapeutic molecules include a protein, a peptide, a polypeptide, an RNA molecule, a peptidomimetic, an siRNA molecule, a gRNA molecule, or a small molecule. Exemplary ROCK inhibitors include, but are not limited to belumosudil (KD025), AT-13148, BA-210, β-elemene, chroman 1, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, and Y-39983.

### B. OXPHOS Inhibitors

OXPHOS is the metabolic pathway by which cells use enzymes to oxidize nutrients, thereby releasing chemical energy in order to produce adenosine triphosphate (ATP). In eukaryotes, this takes place inside mitochondria. Enzymes of the eukaryotic OXPHOS pathway include NADH-coenzyme Q oxidoreductase, succinate-Q oxidoreductase, electron transfer flavoprotein-Q oxidoreductase, Q-cytochrome c oxidoreductase, and cytochrome c oxidase. As used herein the term “OXPHOS inhibitor” refers to a compound that may bind to, decrease, prevent, delay activation, inactivate, desensitize, or downregulate the activity or expression of an OXPHOS metabolic pathway enzyme. Inhibitors of OXPHOS may also include, in certain embodiments, genetically modified versions of an OXPHOS associated enzyme, e.g., versions with altered activity, as well as naturally occurring and antagonists, antibodies, peptides, cyclic peptides, nucleic acids, antisense molecules, ribozymes, small chemical molecules, and the like. Assays for inhibitors include, e.g., expressing an OXPHOS associated enzyme in vitro, in cells, or in cell membranes, applying putative inhibitor compounds, and then determining the functional effects on activity, as described herein.

Test samples or assays comprising OXPHOS associated enzymes that are treated with a potential inhibitor may be compared to a control sample lacking the inhibitor in order to determine the extent of inhibition. Control samples to which a test sample or assay is compared may be assigned a relative protein activity value of 100%. Inhibition of OXPHOS is achieved when the activity value of the test sample relative to the control sample is less than about 80%, including less than about 75%, about 70%, about 65%, about 60%, about 55%, about 50%, about 45%, about 40%, about 35%, about 30%, about 25%, about 20%, about 15%, about 10%, about 5%, and about 1%, including all ranges and values derivable therebetween.

According to the present disclosure, any therapeutic molecule may be used to inhibit OXPHOS. Non-limiting examples of such therapeutic molecules include a protein, a peptide, a polypeptide, an RNA molecule, a peptidomimetic, an siRNA molecule, a gRNA molecule, or a small molecule. An OXPHOS inhibitor of the present disclosure may, in some embodiments, reduce ATP production. In particular embodiments of the present disclosure, an OXPHOS inhibitor may reduce the expression of OXPHOS or oxidation reduction pathway related genes. Non-limiting example of such genes include Cox6c, Mdh1, Acat1, and Glo1.

Any OXPHOS inhibitor may be used according to the embodiments of the present disclosure. Exemplary OXPHOS inhibitors include, but are not limited to, IACS-010759 (IACS-10759), metformin, phenformin, HP661, IM156 (lixumistat, HL156A), BAY 87-2243, VLX600, lonidamine, atovaquone, AG311, Mito-Met₁₀ (norMitoMet), mubritinib, carboxyamidotriazole (CAI), ME344, fenofibrate, deguelin, papaverine, α-TOS, neoantimycin F, ADDA 5, Gboxin, S-Gboxin, oligomycin A, apoptolidin, bedaquiline, DX3-213B, BAY-179, 4-methyl-2-oxovaleric acid (ketoleucine, 4-MOV, KIC), diphenylamine hydrochloride, and carbonylcyanide 3-chlorophenylhydrazone (CCCP), carbonyl cyanide 4-(trifluoromethoxy)phenylhydrazone (FCCP), 3-nitropropionic acid, amobarbital, antimycin A, arsenic trioxide, atpenin A5, aurovertin B, BAM 15, Bz-423, berberine, canagliflozin, calcimycin (A-23187), cyanine5 alkyne (alkyne-Cy5), DX2-201, DX3-234, DX3-235, hydrocortisone, IM176OUT05, malonate, mIBG, Mito-LND (Mito-Lonidamine), Mito-Q, MPTP (1-methyl 4-phenyl 1,2,3,6 tetrahydropyridine), myxothiazol nitric oxide, nefazodone, nonactin, parimifasor (LYC-30937), piericidin A, pioglitazone, pyrvinium, ranolazine, rosiglitazone, rotenone, RTB70, TRC1, OXPHOS-IN-1, SMV-32, stigmatellin, TRAP1-IN-2, TRAP1-IN-1, SCAL-255, SCAL-266, siccanin, tetrathiomolybdate, tiabendazole, and TTFA (thenoyltrifluoroacetone)

### C. Therapeutic and Pharmaceutical Compositions

In certain aspects, the present disclosure provides pharmaceutical and therapeutic compositions comprising a ROCK inhibitor and an OXPHOS inhibitor. In some embodiments, the ROCK inhibitor and/or OXPHOS inhibitors of the present disclosure may be combined with a pharmaceutically acceptable carrier. As used herein, a “pharmaceutically acceptable carrier,” “pharmaceutically acceptable adjuvant,” or “adjuvant” refers to reagents, cells, compounds, materials, compositions, and/or dosage forms that are not only compatible with the ROCK inhibitors, OXPHOS inhibitors, or other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Also included may be an agent that modifies the effect of other agents and is useful in preparing a therapeutic compound or pharmaceutical compound or composition that is generally safe, non-toxic, and neither biologically nor otherwise undesirable. Such an agent may be added to a therapeutic composition or pharmaceutical composition to modify for example the cellular target, cellular localization, or cellular uptake of a ROCK inhibitor or an OXPHOS inhibitor as described herein. Such an agent may include any excipient, diluent, carrier, or adjuvant that is acceptable for pharmaceutical use. Such an agent may be non-naturally occurring, or may be naturally occurring, but not naturally found in combination with other agents in the therapeutic or pharmaceutical composition.

As used herein, a “therapeutic compound” or “therapeutic composition” refers to a composition comprising a ROCK inhibitor and/or an OXPHOS inhibitor of the present disclosure. In some embodiments, a therapeutic composition has the activity of reducing the activity, function, or expression of ROCK. In certain embodiments, a therapeutic composition has the activity of reducing the activity, function, or expression of an OXPHOS metabolic pathway enzyme in a subject as described herein. In one embodiment, the composition is capable of reducing, stabilizing, or eliminating tumor growth or tumor progression in a subject. In another embodiment, the composition is capable of reducing, stabilizing, or eliminating tumor size in a subject. In particular embodiments, a therapeutic composition of the present disclosure is capable of reducing or increasing the expression of genes associated with the oxidation-reduction process, G protein-coupled receptor signaling, Rho GTPase signaling, oxidative phosphorylation, the electron transport chain, nucleotide metabolic processes, lipid metabolic processes, carboxylic acid metabolic processes, regulation of the ERK1/2 cascade, regulation of the mitotic cell cycle, NADH dehydrogenase complex assembly, cAMP-mediated signaling, or the tricarboxylic acid cycle.

A compound or composition of the present disclosure is meant to encompass a composition suitable for administration to a subject, such as a mammal, particularly a human subject. In general, a therapeutic composition is sterile, and preferably free of contaminants that are capable of eliciting an undesirable response within the subject (e.g., the compound(s) in the composition are pharmaceutical grade). Therapeutic compositions may be designed for administration to subjects in need thereof via a number of different routes of administration including oral, intravenous, intraarticular, intraarterial, buccal, rectal, parenteral, intraperitoneal, intradermal, intratracheal, intramuscular, subcutaneous, inhalation, vaginal, intraosseous, trans nasal, injection, microneedle, topical, and transdermal. The appropriate dosage of a composition, as described herein, may be determined based on the type of disease to be treated, the severity and course of the disease, the clinical condition of the individual, clinical history, response to the treatment, and the discretion of the attending physician. In some embodiments, therapeutic compositions provided by the present disclosure may include various “unit doses.” A unit dose is defined as containing a predetermined quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The phrase “effective amount” as used herein refers to a concentration or amount of a therapeutic compound or composition as described herein, a reagent, or another agent, which is effective for producing an intended result, including treatment of cancer as described herein. With respect to the administration of a therapeutic compound as disclosed herein, an effective amount may be any effective range or concentration. The exact dose will depend on the purpose of the treatment, and one of skill in the art will be able to determine such a dose using techniques known in the art.

Therapeutic compounds or compositions may be provided to a subject in a single dose or multiple doses and as such provided in single-dose or multi-dose containers, such as sealed ampules or vials. Such containers may be sealed to preserve sterility of the composition until use. In general, compositions as described herein may be stored as suspensions, solutions, or emulsions in oily or aqueous vehicles. Alternatively, such a composition may be stored in a freeze-dried condition requiring only the addition of a sterile liquid carrier immediately prior to use. In some embodiments the therapeutic compounds or compositions may be stored in tablet form.

In some embodiments, an inhibitor, pharmaceutical composition, or therapeutic composition of the present disclosure may administered at an effective amount of about 1 mg/kg to about 2500 mg/kg body weight, about 10 mg/kg to about 2000 mg/kg body weight, about 50 mg/kg to about 1750 mg/kg body weight, about 100 mg/kg to about 1500 mg/kg body weight, about 200 mg/kg to about 1200 mg/kg body weight, about 100 mg/kg to about 800 mg/kg body weight, about 100 mg/kg to about 600 mg/kg body weight, about 200 mg/kg boy weight to about 500 mg/kg body weight, about 200 mg/kg to about 400 mg/kg body weight, about 0.5 mg/kg to about 2500 mg/kg body weight, about 10 mg/kg to about 2000 mg/kg body weight, about 50 mg/kg to about 1750 mg/kg body weight, about 200 mg/kg to about 1000 mg/kg body weight, about 200 mg/kg to about 800 mg/kg body weight, about 400 mg/kg to about 600 mg/kg body weight, about 0.5 mg/kg to about 20 mg/kg body weight, about 0.5 mg/kg to about 15 mg/kg body weight, about 0.5 mg/kg to about 10 mg/kg body weight, about 0.5 mg/kg to about 9 mg/kg body weight, about 0.5 mg/kg to about 8 mg/kg body weight, about 0.5 mg/kg to about 7 mg/kg body weight, about 0.5 mg/kg to about 6 mg/kg body weight, about 0.5 mg/kg to about 5 mg/kg body weight, about 0.5 mg/kg to about 4 mg/kg body weight, about 0.5 mg/kg to about 3 mg/kg body weight, about 0.5 mg/kg to about 2 mg/kg body weight, or about 0.5 mg/kg to about 1.5 mg/kg body weight, including all ranges and values derivable therebetween. In one embodiment, an effective amount of an inhibitor as described herein may be represented as a daily effective amount, a twice daily effective amount, a bi-daily effective amount, a weekly effective amount, or a monthly effective amount.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

As used herein, “subject” or “patient” refers to animals, including humans, who are treated with the inhibitors, therapeutic compounds, or compositions or in accordance with the methods described herein. For diagnostic or research applications, a wide variety of mammals may be suitable subjects, including rodents (e.g., mice, rats, hamsters), rabbits, primates, and swine, such as inbred pigs. In particular embodiments, a subject in need of therapy may be any subject who comprises a cell that exhibits a mutation in a subunit of a SWI/SNF chromatin remodeling complex as described herein. In another embodiment, the subject may be afflicted with or at risk of developing a disease or condition associated with a mutation in a subunit of a SWI/SNF chromatin remodeling complex as described herein. The subunit of a SWI/SNF chromatin remodeling complex, in some embodiments, may be ARID1A or SMARCA4. Non-limiting examples of diseases or conditions that a subject of the present disclosure may be afflicted with or at risk of developing include lung cancer, non-small cell lung cancer (NSCLC), small cell lung cancer (SCLC), brain cancer, glioblastoma, medulloblastoma, skin cancer, melanoma, pancreatic cancer, colorectal cancer, appendiceal cancer, hematopoietic cancer, B-cell lymphoma, leukemia, myeloma, breast cancer, head and neck cancer, prostate cancer, kidney cancer, bladder cancer, liver cancer, esophageal cancer, stomach cancer, thyroid cancer, small bowel adenocarcinoma, hepatobiliary cancer, gynecological cancer, cervical cancer, uterine cancer, and ovarian cancer.

In certain embodiments, a subject of the present disclosure may comprise a cancer that is resistant to chemotherapy, immunotherapy, or a small molecule inhibitor. Chemotherapy, immunotherapy, and small molecule inhibitors for the treatment of cancer are known in the art and any such agent may be used according to the embodiments of the present disclosure. Non-limiting examples of such agents include alkylating agents, antimetabolites, anti-tumor antibiotics, topoisomerase inhibitors, mitotic inhibitors, immune checkpoint inhibitors, immunomodulators, cytokines, monoclonal antibodies, CAR T therapy, oncolytic virus therapy, cancer vaccines, adoptive cell therapy, targeted antibodies, kinase inhibitors, epigenetic modulators, enzyme inhibitors, and chemokine receptors antagonists. In particular embodiments, a subject of the present disclosure may comprise a cancer that is resistant to an inhibitor of KRAS. Non-limiting example of inhibitors of KRAS include sotorasib, adagrasib, MRTX1133, ARS-3248, ARS-853, ARS-1620, opnurasib, GDC-6036, RG6330, D-1553, BPI-421286, GH35, BEBT-607, JAB-21000, BI-2865, BI-2493, and RMC-6236.

A composition, as described herein, may include, in particular embodiments, a combination of therapeutic agents. In some embodiments, a composition as described here may be administered as a single composition or as more than one composition. Different compositions as provided herein, in certain embodiments, may be administered by the same route of administration or by different routes of administration.

A pharmaceutical composition of the present disclosure may comprise, in certain embodiments, a ROCK inhibitor and/or an OXPHOS inhibitor. Any ROCK inhibitor and any OXPHOS inhibitor known in the art may be used in a pharmaceutical composition of the present disclosure. Non-limiting examples of such ROCK inhibitors include belumosudil (KD025), AT-13148, BA-210, β-elemene, chroman 1, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, and Y-39983. Non-limiting examples of such OXPHOS inhibitors include IACS-010759 (IACS-10759), metformin, phenformin, HP661, IM156 (lixumistat, HL156A), BAY 87-2243, VLX600, lonidamine, atovaquone, AG311, Mito-Met₁₀ (norMitoMet), mubritinib, carboxyamidotriazole (CAI), ME344, fenofibrate, deguelin, papaverine, α-TOS, neoantimycin F, ADDA 5, Gboxin, S-Gboxin, oligomycin A, apoptolidin, bedaquiline, DX3-213B, BAY-179, 4-methyl-2-oxovaleric acid (ketoleucine, 4-MOV, KIC), diphenylamine hydrochloride, and carbonylcyanide 3-chlorophenylhydrazone (CCCP), carbonyl cyanide 4-(trifluoromethoxy)phenylhydrazone (FCCP), 3-nitropropionic acid, amobarbital, antimycin A, arsenic trioxide, atpenin A5, aurovertin B, BAM 15, Bz-423, berberine, canagliflozin, calcimycin (A-23187), cyanine5 alkyne (alkyne-Cy5), DX2-201, DX3-234, DX3-235, hydrocortisone, IM176OUT05, malonate, mIBG, Mito-LND (Mito-Lonidamine), Mito-Q, MPTP (1-methyl 4-phenyl 1,2,3,6 tetrahydropyridine), myxothiazol nitric oxide, nefazodone, nonactin, parimifasor (LYC-30937), piericidin A, pioglitazone, pyrvinium, ranolazine, rosiglitazone, rotenone, RTB70, TRC1, OXPHOS-IN-1, SMV-32, stigmatellin, TRAP1-IN-2, TRAP1-IN-1, SCAL-255, SCAL-266, siccanin, tetrathiomolybdate, tiabendazole, and TTFA (thenoyltrifluoroacetone).

In certain embodiments, a ROCK inhibitor or OXPHOS inhibitor of the present disclosure may include a therapeutic peptide or nucleic acid. Any therapeutic peptide or nucleic acid known in the art to reduce the activity, function, or expression of ROCK or OXPHOS may be used according to the embodiments of the present disclosure. In particular embodiments, the therapeutic peptide or therapeutic nucleic acid may function in or encode a protein or small RNA molecule associated with reduced ROCK or OXPHOS activity, expression, or function. A pharmaceutical composition of the present disclosure may comprise, in some embodiments, a targeting molecule or a nanoparticle for delivery of a therapeutic peptide or nucleic acid. In one embodiment, the targeting molecule or nanoparticle may be cell-specific or tissue-specific. Numerous such targeting molecules and nanoparticles are known in the art and any such targeting molecule may be used according to certain embodiments of the present disclosure. In certain embodiments, a composition of the present disclosure may be modified with or conjugated to a peptide, a protein, a colloidal molecule, or a polymer to facilitate delivery or adsorption. The pharmaceutical composition of the present disclosure, in some embodiments, may be serum-free, endotoxin-free, or sterile.

A peptide or polynucleotide molecule for use according to the compositions of the present disclosure may, in some embodiments, be a recombinant peptide or nucleic acid. As used herein, the term “recombinant” refers to a polynucleotide molecule, protein, or cell that is not naturally present, or is not naturally present in the same form or structure and was created by human intervention. In one embodiment, a recombinant polynucleotide may be a DNA molecule or may be an RNA molecule. A recombinant polynucleotide molecule or a recombinant polypeptide molecule or protein may comprise, in certain embodiments, a combination of two or more polynucleotide or polypeptide sequences that do not naturally occur together in the same manner, such as a polynucleotide molecule or protein that comprises at least two polynucleotide or protein sequences that are operably linked but heterologous with respect to each other. As used herein the term “heterologous” refers to a polynucleotide molecule or protein that is not naturally present or is not naturally present in the same form or structure and was created by human intervention. For example, a heterologous polynucleotide molecule or protein may not naturally occur in the cell being transformed or may be expressed in a manner or genomic context that differs from the natural expression pattern or genomic context found in the cell being transformed. The heterologous polynucleotide molecule or protein, in some embodiments, may be overexpressed in the cell being transformed. In certain embodiments, a recombinant polynucleotide molecule, protein, construct, or vector may comprise any combination of two or more polynucleotide or protein sequences in the same molecule which are heterologous to one another, such that the combination is man-made and not normally found in nature. As used herein, the phrase “not normally found in nature” means not found in nature without human intervention. A recombinant polynucleotide or protein molecule, may comprise, for example, polynucleotide or protein sequences that are separated from other polynucleotide or protein sequences that exist in proximity to each other in nature. A recombinant polynucleotide or protein molecule may also comprise, for example, polynucleotide or protein sequences that are adjacent to or contiguous with other polynucleotide or protein sequences that are not naturally in proximity with each other. Such a recombinant polynucleotide molecule, protein, or expression construct may also refer to a polynucleotide or protein molecule or sequence that has been genetically engineered or constructed outside of a cell. For example, a recombinant polynucleotide molecule may comprise any engineered or man-made plasmid, vector, or expression construct, and may include a linear or circular DNA molecule. Such plasmids, vectors, and expression constructs may comprise, for example, various maintenance elements including, but not limited to, a heterologous promoter sequence, a prokaryotic origin of replication, or a selectable marker.

In certain aspects, a therapeutic composition of the present disclosure may comprise a ROCK inhibitor or an OXPHOS inhibitor of the present disclosure and a second therapeutic agent or a detectable label. Non-limiting example of therapeutic agents or detectable labels that may be used according to the present disclosure include a chemotherapeutic agent, an immunotherapeutic agent, a mitochondrial therapeutic agent, a neurotherapeutic agent, a metabolic therapeutic agent, or a radiotherapeutic agent. Non-limiting examples of detectable labels that may be used according to embodiments of the present disclosure include a paramagnetic ion, a radioactive isotope, a fluorochrome, an NMR-detectable agent, or an X-ray imaging agent. As used herein, the term “label” refers to a directly or indirectly detectable compound or composition that is conjugated directly or indirectly to the composition to be detected. In certain embodiments, an inhibitor, a polynucleotide molecule, protein, or cell may be labeled to generate a labeled composition. In particular embodiments, labeled compositions also include sequences which are conjugated a polynucleotide molecule that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition that is detectable. Labels may be suitable for small scale detection or for high-throughput screening. As such, suitable labels include, but are not limited to radioisotopes, fluorochromes, chemiluminescent compounds, dyes, and proteins, including enzymes. Labels may be simply detected or may be quantified. In certain embodiments, labels that may be quantified provide numerically reportable value. In luminescence or fluorescence assays, the detectable response may be generated directly using a luminophore or fluorophore associated with an assay component involved in binding, or indirectly using a luminophore or fluorophore associated with another (e.g., reporter or indicator) component.

In certain embodiments, the compositions and methods for treating an individual described herein may be combined with any other composition or method of treatment known in the art. The compositions and methods may be administered in any suitable manner known in the art. For example, a first and a second therapeutic agent or inhibitor may be administered sequentially (at different times) or concurrently (at the same time). In some aspects, a first and a therapeutic agent or inhibitor may be administered in separate compositions. In certain embodiments, a first and a second cancer treatment or inhibitor may be administered in the same composition.

Non-limiting examples of additional treatment modalities that may be included in combination with the compositions and methods provided herein include a therapeutic agent or surgery. In specific embodiments, the methods and compositions of the present disclosure may be combined with other therapies directed towards the treatment of cancer as described herein.

The term “about” is used to indicate that a value includes the standard deviation of the mean for the device or method being employed to determine the value. The use of the term “or” in the claims is used to mean “and/or” unless explicitly indicated to refer to alternatives only or the alternatives are mutually exclusive. When used in conjunction with the word “comprising” or other open language in the claims, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps. Similarly, any system or method that “comprises,” “has,” or “includes” one or more components is not limited to possessing only those components and covers other unlisted components.

Other objects, features, and advantages of the present disclosure are apparent from detailed description provided herein. It should be understood, however, that the detailed description and any specific examples provided, while indicating specific embodiments of the disclosure, are given by way of illustration only, since various changes and modifications within the spirit and scope of the disclosure will become apparent to those skilled in the art from this detailed description. Any embodiment of the present disclosure may be used in combination with any other embodiment described herein.

All references herein are incorporated herein by reference in their entirety.

### EXAMPLES

The following examples are included to illustrate embodiments of the present disclosure. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1: Functional Genomics Identifies the ROCK Inhibitor KD025 as a Novel, Effective Combinatorial Agent With OXPHOS Inhibition

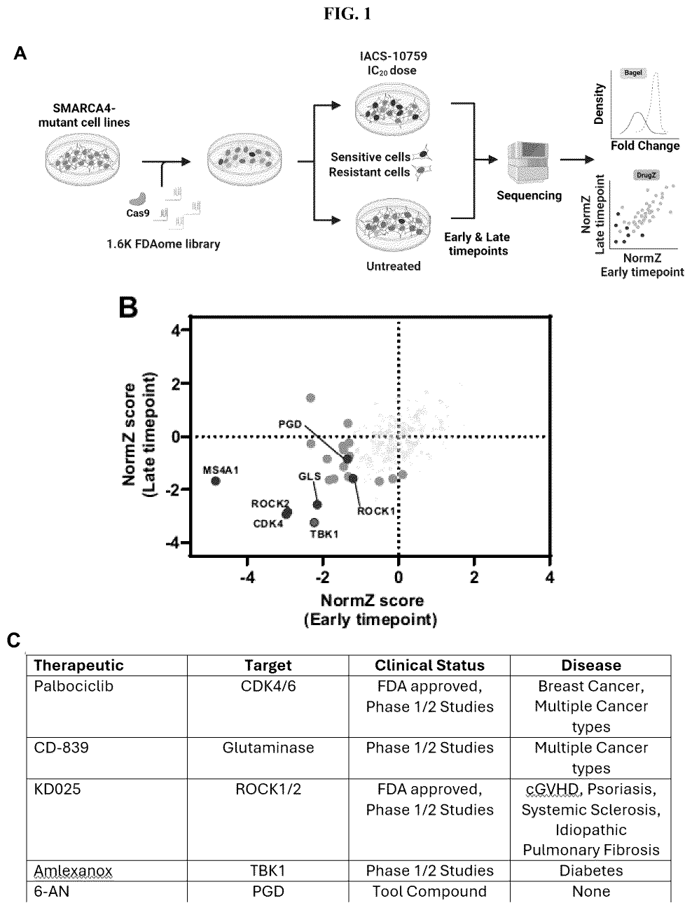

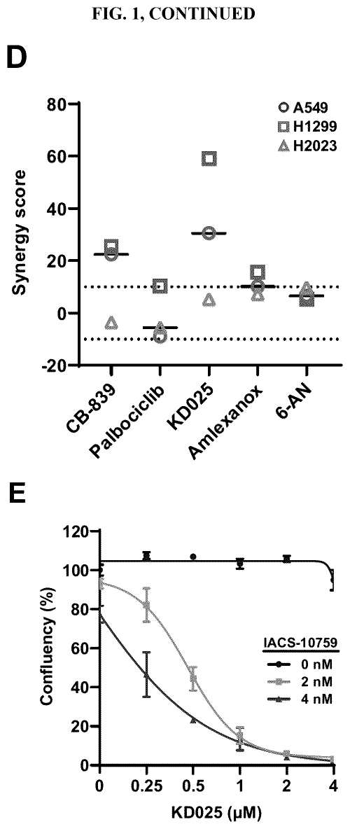

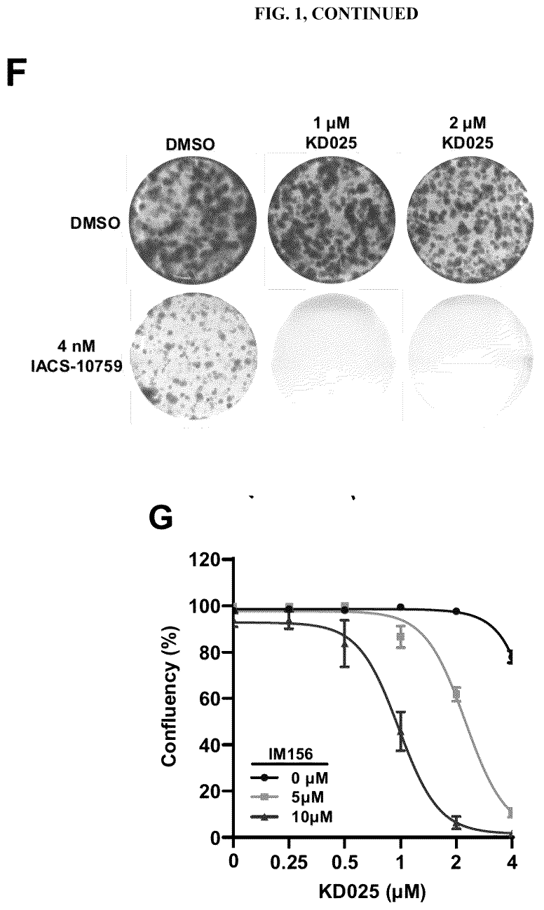

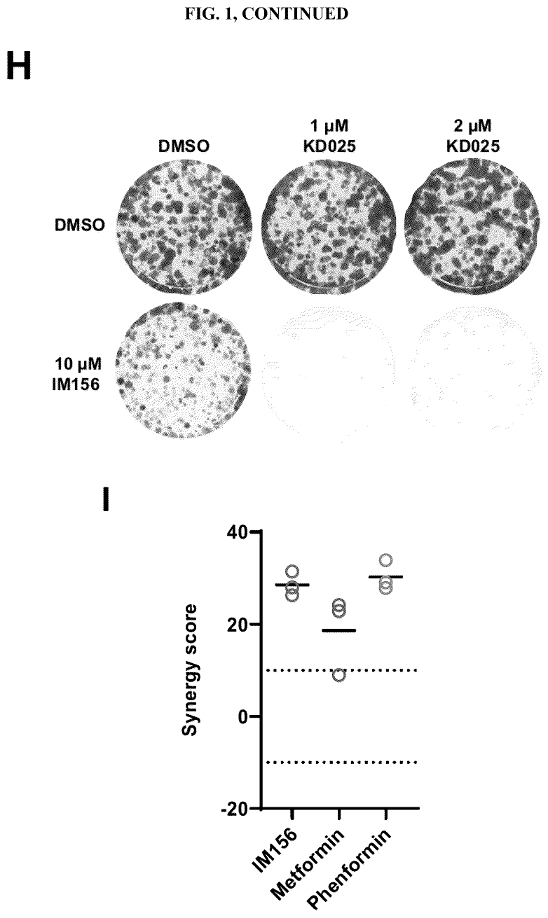

As a first step in a broad and systematic approach aimed at discovering clinically relevant agents that combine well with OXPHOS inhibition, CRISPR screens were performed using a focused “FDAome” library that contains 1607 gRNAs targeting 200 protein coding genes having gene products with FDA approved therapeutics or therapeutics in clinical testing. The CRISPR screens were carried out in three cell lines with damaging SMARCA4 mutations, as these are more sensitive to IACS-10759 compared to SMARCA4 wild-type cells due to their dependance on OXPHOS. The IC₂₀ (inhibitory dose corresponding to 20% growth inhibition) was used for IACS-10759 treatment as this dose is well tolerated in patients. Each cell line was transduced with the lentiviral-based FDAome CRISPR library at a low multiplicity of infection (MOI) (<0.3), followed by selection with puromycin. Cells were treated with either dimethyl sulfoxide (DMSO) as a control or with IACS-10759 for the duration of the experiment. Cells collected after puromycin selection and seeding were referenced as time=0 (T0), and cells were cultured for at least 10 population doublings and collected at both early and late time points. Genomic DNA was extracted and barcode labeled. Final PCR products were submitted for deep-sequencing and analyses (FIG. 1, Panel A). Processing of sequencing reads for each screen assured an adequate depth and distribution (>1000-fold of gRNA numbers) for subsequent bioinformatic analysis. Next, analysis was performed using the BAGEL algorithm to calculate a Bayes factor for each gene and Pearson's correlation coefficients were calculated based on the Bayes factor distributions. Notably, H1299, A549, and H2023 screens had a high correlation (correlation index>0.8) among the different groups indicating that the results of these 3 screens were of comparable quality. Precision-recall curves were also used to evaluate the performance of each screen and a value>0.8 was routinely observed. This metric is commonly used to determine a quality screen. Changes in 50 essential and 50 nonessential control genes included in the FDAome library were also compared from the starting reference time point (T0) to the early and late time points. Overall, sgRNAs distributions targeting essential genes were reduced, while those targeting nonessential genes did not change regardless of the time point analyzed, indicating that the CRISPR screens worked well in each cell line. Taken together, these results demonstrate that all three screens were of high quality and performance allowing for reliable identification of co-essential genes to OXPHOS inhibition.

Focusing on the identification of genes whose depletion caused fitness defects and reduced viability with IACS-10759, a normalized sgRNA depletion score was calculated for each gene using DrugZ by comparing the IACS-10759-treated group and the DMSO-treated group in each cell line at both early and late time points. Genes from each cell line were ranked by their DrugZ scores. High-confidence hits were determined at each timepoint by including genes with a p-value<0.05 for each cell line or gene in at least two cell lines with a p-value threshold of <0.10. This analysis identified a total of 59 unique genes across all three cell lines. Out of this list, genes were prioritized that were depleted at both early and late timepoints in at least one cell line that also had multiple sgRNAs (n>3) depleted in the IACS-10759-treated group compared to the DMSO-treated group (FIG. 1, Panel A, Panel B, and Panel C). In total, 5 genes were identified as potential synthetic lethal targets in IACS-10759-treated cancer cells. Notably, the top hit from this analysis was PGD, a metabolizing enzyme essential for generating NADPH for redox homeostasis, included in the FDAome library for quality control, as previous CRISPR screens demonstrated PGD as a synthetic lethal target to OXPHOS deficiency. Other lead candidate co-essential genes included TBK1, CDK4, GLS, and ROCK1/ROCK2. TBK1 is a non-conical IKK serine/threonine kinase that regulates NFKB involved in innate immunity with additional diverse roles in autophagy, proliferation, apoptosis, and glucose metabolism. CDK4 is a serine/threonine kinase involved in G1 cell cycle phase progression. CDK4/6 inhibition is synthetic lethal in SMARCA4-mutant lung cancer. GLS is mitochondrial enzyme that promotes the breakdown of glutamine to glutamate to fuel the tricarboxylic (TCA) cycle via anaplerosis or fatty acid synthesis via reductive carboxylation. ROCK1 and ROCK2 are serine/threonine kinases that regulate actin cytoskeletal rearrangements involved in cancer cell migration, motility, and invasion as well as glucose homeostasis. These targets were prioritized for further analysis by identifying therapeutics that are FDA approved or in advanced clinical development (FIG. 1, Panel C).

To determine whether therapeutics targeting candidate co-essential genes sensitize cells to OXPHOS inhibition, a combinatorial drug screening strategy was employed with IACS-10759 in A549, H1299, and H2023 SMARCA4-mutant cell lines. Five-day growth assays were performed with a matrix titration of several doses of each agent alone or in combination. The statistical significance of growth inhibition was determined by the combination of IACS-10759 and each respective therapeutic by determining a consensus synergy score (>10 indicates synergy) by Bliss estimation using SynergyFinder (REF) (FIG. 1, Panel D). This approach revealed that the combination of IACS-10759 with 6-aminonicotinamide (6-AN), a cell-permeable compound that suppresses PGD activity by competing with nicotinamide for NAD⁺/NADP⁺, was additive/weakly synergistic for suppressing cell growth, further demonstrating the predictability of the screens. Overall, the results of the combination drug screen revealed that pharmacological inhibition with KD025, a ROCK inhibitor, was most effective in combination with IACS-10759. These two inhibitors showed high synergism in all three SMARCA4-mutant lung cancer cell lines (FIG. 1, Panel E). Further, this combination completely suppressed cell growth in all three cell lines, while individual drug exposure had little effect on cell growth at the doses examined. (FIG. 1, Panel E). This response was confirmed by performing 12 to 14-day clonogenic assays, which similarly showed the robust and highly significant synergy of the KD025 and IACS-10759 combination (FIG. 1, Panel F). These studies were designed using sub-therapeutic doses of IACS-10759 that avoid the dose limiting toxicity of this compound observed in the clinic. It was next determined whether the repertoire of OXPHOS inhibitors could be expanded to maximize the potential of KD025 as a suitable combinatorial agent in the clinic.

Importantly, synergistic inhibition on cell growth was maintained when other clinically available small-molecule agents targeting OXPHOS, such as IM156, metformin, and phenformin were used in combination with KD025 in H1299 cells (FIG. 1, Panel H, Panel I). In line with these pharmacologic results, genetic perturbation of ROCK1/2 by shRNA knockdown in H1299 cells treated with IACS-10759 also caused a synergistic anti-proliferative response (data not shown). Taken together, these results demonstrate that ROCK inhibition has synergistic anti-tumor activity in combination with OXPHOS inhibition.

### Example 2: Rock Blockade Combined With OXPHOS Inhibition Causes Cell Cycle Growth Arrest, Apoptosis, and Severe Energy Deficiency

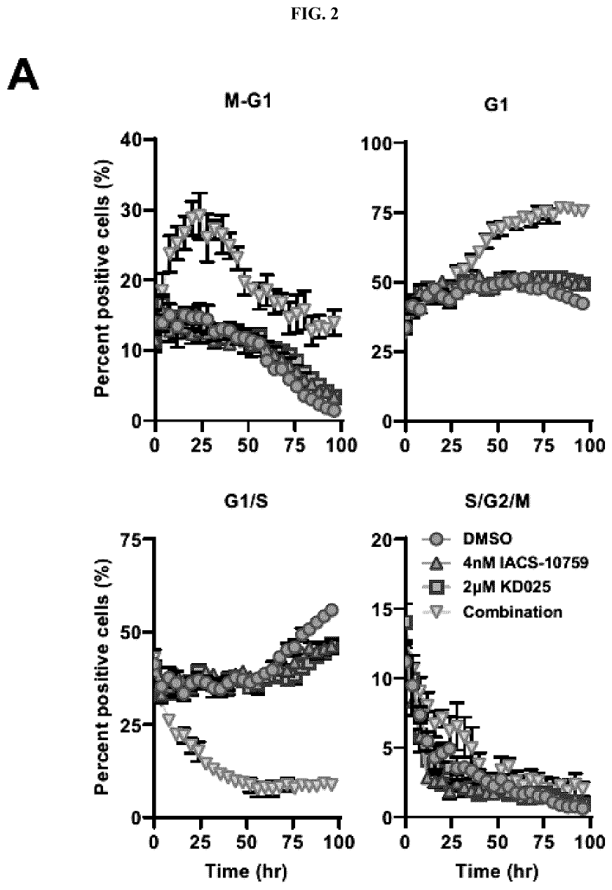

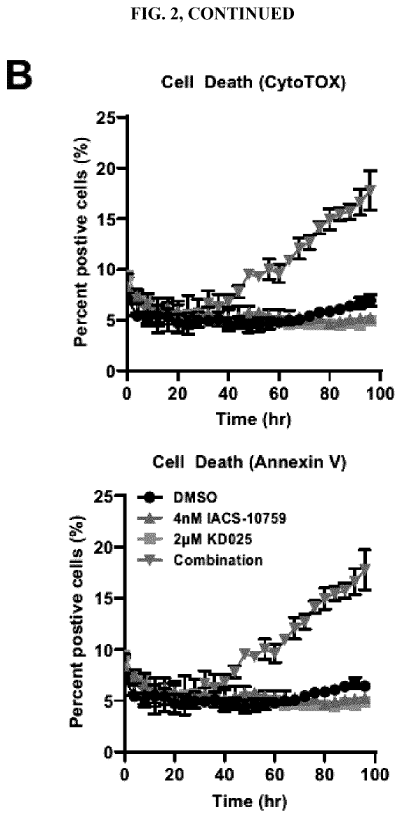

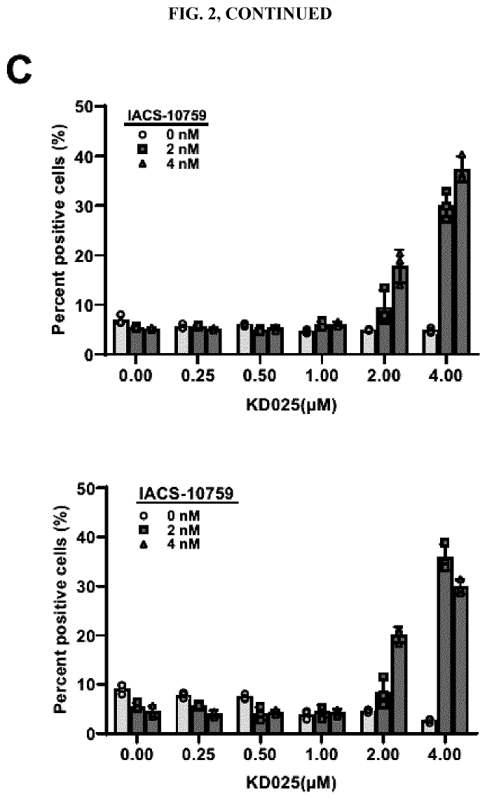

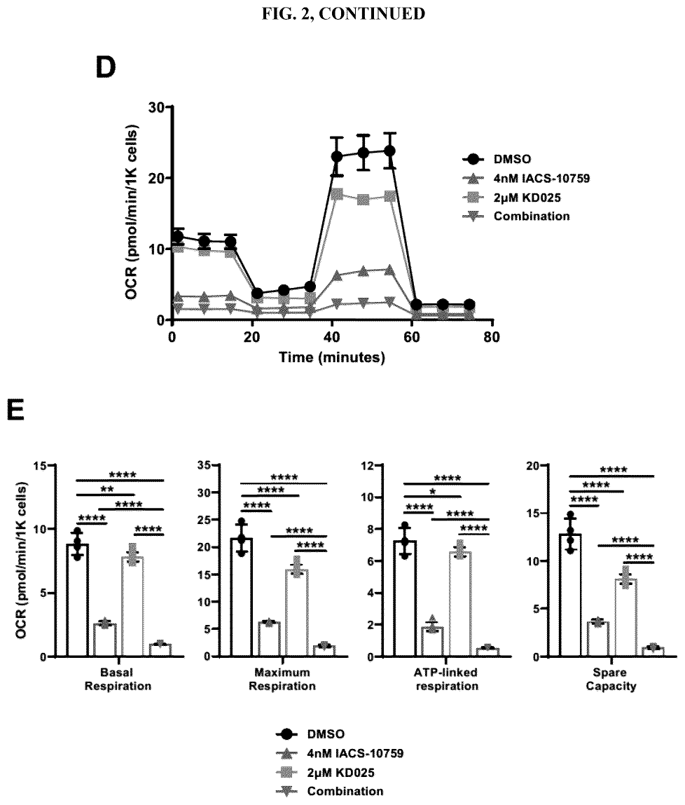

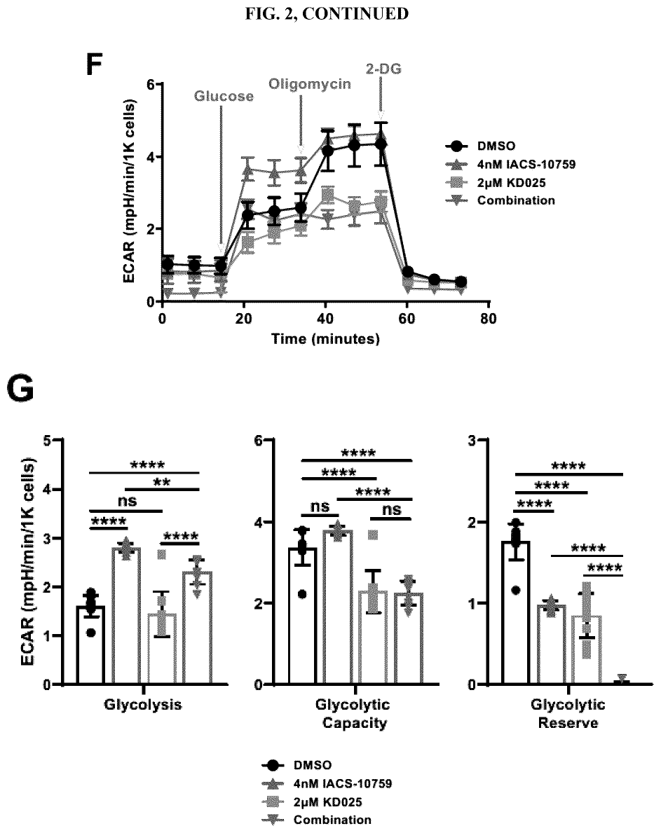

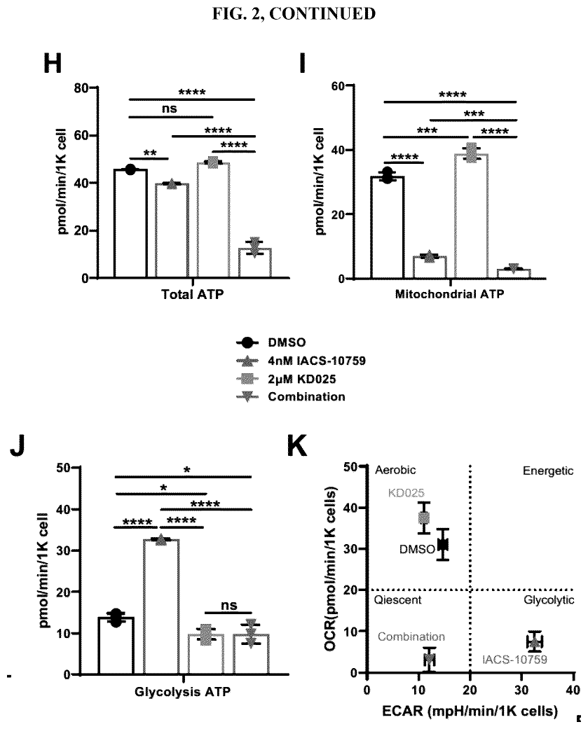

To gain additional insight into the strong synergistic inhibitory action between KD025 and IACS-10759, the cell cycle reporter (FUCCI) was used to monitor different phases of the cell cycle in real-time in H1299 cells (FIG. 2, Panel A). Treatment with either KD025 or IACS-10759 alone had no significant effect on the cell cycle phase distribution compared to vehicle-treated cells (FIG. 2, Panel A). In contrast, combination of KD025 and IACS-10759 increased the percentage of cells in M-G1 transition and the G1 cell cycle phase over the time with a concomitant decrease in the percentage of cells in the G1/S cell cycle phase (FIG. 2, Panel A), indicating that these cells go into a M-G1/G1 phase cell cycle arrest. Quantification of cell death demonstrated that the inhibition of OXPHOS and ROCK induces cell death at later time points, particularly around 48 hours in cells expressing oncogenic KRAS, while having no effect in KRAS-independent cells or HBECs. Furthermore, combination of KD025 and IACS-10759 induced apoptotic cell death as determined by Annexin V and Cytotox Green assays, while individually, each compound had no effect (FIG. 2, Panel B and Panel C). Together, these data demonstrate that the potent synergistic combination of KD025 and IACS-10759 is synthetic lethal and detrimental to SMARCA4-mutant lung cancer cell survival in vitro.

Given that SMARCA4-mutant cells lung cancer cells depend on elevated OXPHOS activity for survival, the IACS-10759 and KD025 combination was profiled in H1299 cells for its effect on mitochondrial respiration and glycolytic capacity using the OCR and ECAR Seahorse assays. Consistent with previous results, it was observed that even with low dose IACS-10759, H1299 cells had lower basal, ATP-linked, maximum respiration, and spare capacity rates that differed significantly from vehicle-control cells (FIG. 2, Panel D and Panel E). Interestingly, KD025 also had lower mitochondrial respiration, in particular a reduced maximum respiration, and spare capacity rate, although the effect was more modest when compared to IACS-10759. Importantly, combination of both compounds led to an even greater reduction of mitochondrial respiration rates compared to each compound individually (FIG. 2, Panel D and Panel E). IACS-10759 caused a significant increase in glycolysis as demonstrated by a reduction in glycolytic reserves (FIG. 2, Panel F and Panel G), showing an adaptive dependency on glycolysis (i.e., Warburg effect) due to suppressed OXPHOS. Glycolysis remained unchanged by KD025, but the glycolytic capacity and glycolytic reserves were reduced when compared to vehicle-treated cells (FIG. 2, Panel F and Panel G). The effects on glycolysis, glycolytic reserves, and glycolytic capacity by each compound individually (adaptive vs inhibition) led to an overall suppression with combination therapy, demonstrating that intact ROCK1/2 activity is critical to metabolic adaptation upon OXPHOS inhibition.

Consistent with this data, total cellular ATP production was dramatically reduced by the combination of IACS-10759 and KD025, while each compound individually had little to no effect on ATP production rates (FIG. 2., Panel H). Further analysis by Seahorse revealed that the while the proportion of ATP produced by mitochondria was suppressed by IACS-10759, there was a compensatory increase in the glycolytic ATP production rates consistent with ECAR (FIG. 2, Panel I). In contrast, no significant effect of KD025 on mitochondrial or glycolytic ATP production rates was observed. The compensatory increase in ATP production by IACS-10759, however, was blocked with combination treatment. This is likely due to the reduced glycolytic capacity and glycolytic reserves observed with KD025 treatment in H1299 cells (FIG. 2, Panel I). IACS-10759 treated H1299 cells derived almost all of their ATP from glycolysis, displaying a glycolytic index (GI) consistent with the Warburg effect, that in turn was suppressed in combination with KD025 (FIG. 2, Panel J).

### Example 3: Metabolic Rewiring of Glycolysis by IACS-10759 Is Impaired Upon ROCK Inhibition

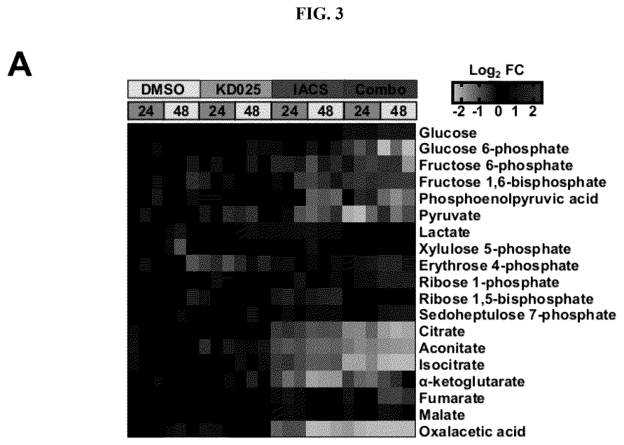

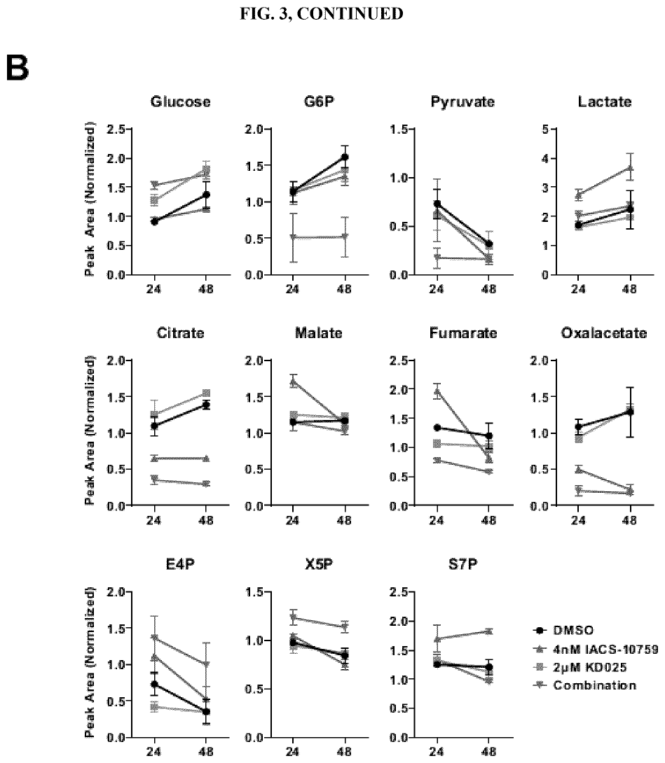

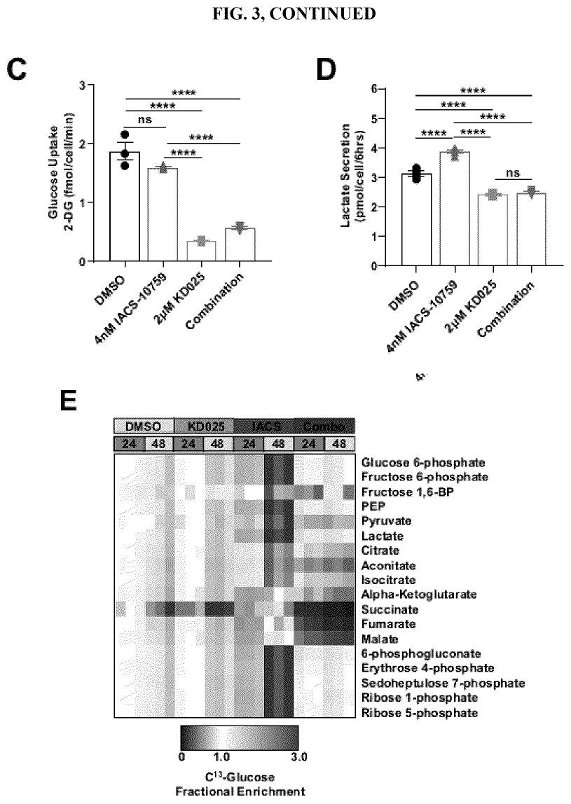

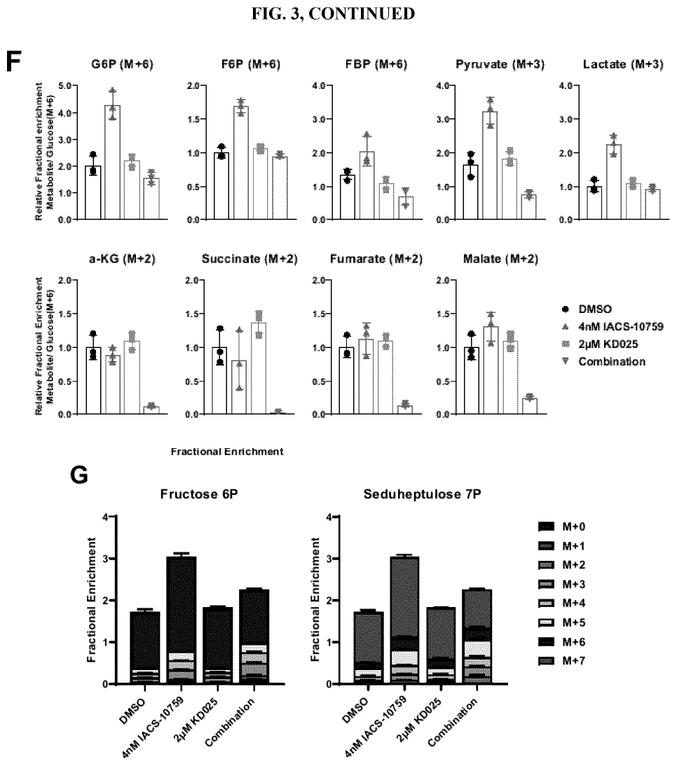

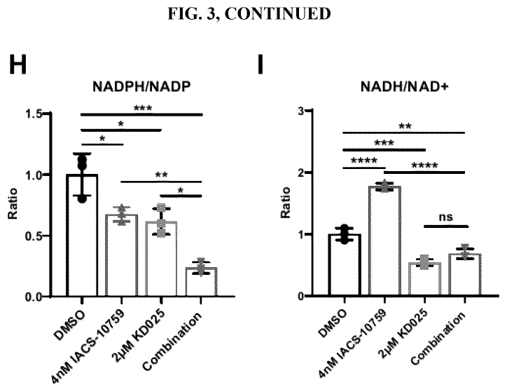

Cancer cells use several carbon sources to fuel glycolysis and the tricarboxylic acid (TCA) cycle to c sustain energy production (i.e., ATP) and the biosynthetic needs for cell survival. The primary carbon sources utilized by cancer cells are glucose and glutamine. To understand how the IACS-10759/KD025 combination impacts cancer cell metabolism, the steady-state abundance of over 300 metabolites was profiled in H1299 cells using gas chromatography/mass spectrometry (GC/MS). The combination of IACS-10759 and KD025 caused a profound rewiring of the metabolic landscape after only 24 and 48 hours compared to treatment with each agent alone. Notably, analysis of significant metabolites showed enrichment of key pathways involved energy metabolism, including the TCA cycle, glycolysis/gluconeogenesis, the pentose phosphate pathway, and pyruvate metabolism, following treatment with IACS-10759 either alone or in combination with KD025. Prompted by this observation and the ECAR results, further analysis was focused on metabolites involved in glycolysis and the TCA cycle. Overall, a dramatic rewiring of glycolysis and the TCA cycle was observed in H1299 cells exposed to the IACS-1059/KD025 combination after 24 and 48 hrs (FIG. 3, Panel A). Several glycolytic metabolites were depleted in the IACS/KD025 combination group compared to the single agent and control groups downstream of glucose, such as glucose-6-phosphate (G6P), fructose-6-phosphase (F6P), and pyruvate (FIG. 3, Panel A and Panel B). Surprisingly, an accumulation of glucose was observed in the IACS/KD025 combination group (FIG. 3, Panel A and Panel B). This effect also observed in cell treated with KD025 alone, demonstrating blockade at the glucose to glucose-6-phosphate rate-limiting step. In contrast, intracellular lactate levels accumulated with IACS-10759 treatment, confirming that OXPHOS inhibition induces the Warburg effect. The increase in lactate, however, was blocked by the IACS/KD025 combination (FIG. 3, Panels A-C). To confirm these results, secretion of extracellular lactate in the media was measured and it was observed that lactate concentration increases in the media following IACS-10759 treatment. This increase was similarly blocked by the IACS/KD025 combination (FIG. 3, Panel D). In addition, KD025 treatment alone and in combination with IACS-10759 resulted in decreased glucose uptake, demonstrating that KD025 inhibits glycolysis likely through multiple-rate limiting steps (FIG. 3, Panel E).

To further support that ROCK inhibition induces an IACS-10759 metabolic shift from consuming glucose, stable isotope tracer analysis was performed using ¹³C₆-glucose (FIG. 3, Panel F). Surprisingly, isotope tracing showed that intracellular ¹³C₆-glucose was decreased in H1299 cells upon IACS treatment, an effect that was rescued with the IACS/KD025 combination (FIG. 3, Panel G). Isotope tracing was reduced in the IACS/KD025 combination for downstream glycolytic metabolites including glucose-6-phosphate (G6P), fructose-6-phosphate (F6P), fructose 1,6-bisphosphate (FBP), and pyruvate (FIG. 3, Panel G). The glycolytic metabolic flux was also measured by determining the ratio of G6P(M+6), pyruvate(M+3), and lactate(M+3) to intracellular ¹³C₆-glucose(M+6). Indeed, IACS-10759 treatment led to an overall increase in the metabolic flux through glycolysis from ¹³C6-glucose to G6P, pyruvate, and lactate that was suppressed by the IACS/KD025 combination (FIG. 3, Panel H and Panel J). These data demonstrate that ROCK inhibition induces metabolic reprogramming that suppresses glucose utilization, depriving cells treated with OXPHOS inhibitors an essential adaptive fuel source for survival.

### Example 4: Phosphoproteomics Links Energy Loss to Metabolic Stress and cell Cycle Growth Arrest in IACS-10759/KD025 Combination

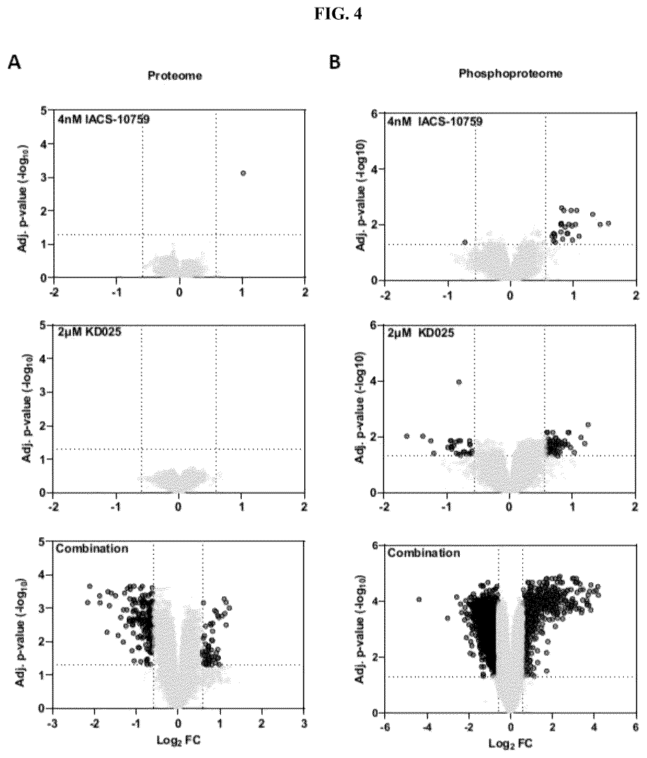

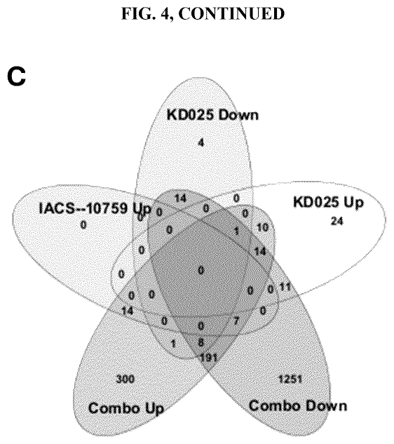

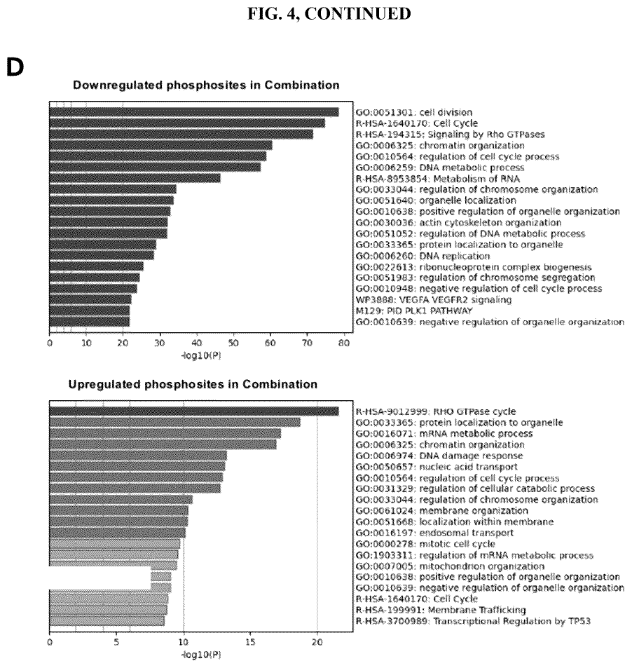

Acute changes in phosphorylation and protein expression were analyzed in H1299 lung cancer cells treated with DMSO, KD025, IACS-10759, and the combination by mass spectrometry-based proteomic and phosphoproteomic analyses. 55,376 phosphopeptides corresponding to 17,539 phosphorylation sites on 4,474 proteins were quantified across all four treatment groups. The proteomic and phosphoproteomic data were of high quality, as revealed by Pearson's correlation, principal component analysis, and hierarchical clustering. Of the 55,376 phosphopeptides analyzed 87 were changed by treatment with IACS-10759, 296 were changed by treatment KD025 and 9,927 were changed by treatment with a combination of IACS-10759 and KD025 (FIG. 4, Panel A and Panel B). FIG. 4, Panel C shows the overlap of phosphosites for IACS-10759, KD025, and the combination. Motif analysis of significantly upregulated and downregulated phosphosites in the IACS-10758/KD025 combination demonstrates that Rho GTPase signaling and cell cycle regulated processes are significantly altered following combination treatment (FIG. 4, Panel D).

### Example 5: Evaluation of Anti-Tumor Activity of the KD025 and IACS-10759 Combination In Vivo

The anti-tumor effect of IACS-010759 and KD025 alone and in combination was evaluated in in vivo mouse xenograft models of lung cancer. Mice bearing H1299 tumor xenografts were treated with daily oral administration of 300 mg per kg body weight of KD025 and 1 mg kg body weight IACS-10759, both of which are clinically relevant doses.

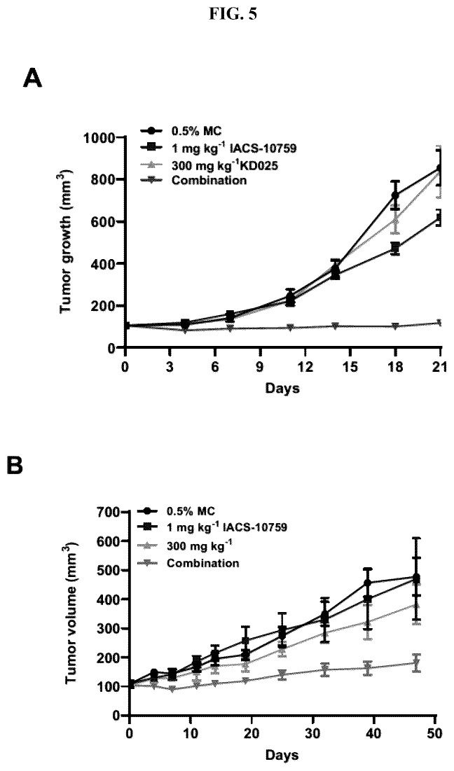

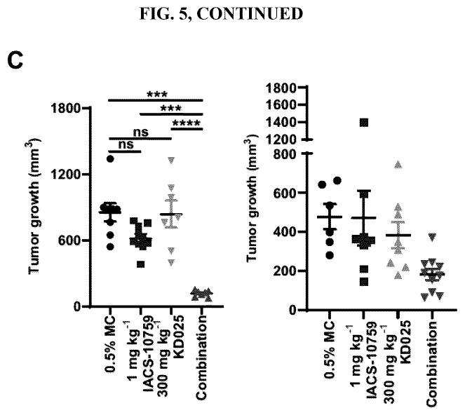

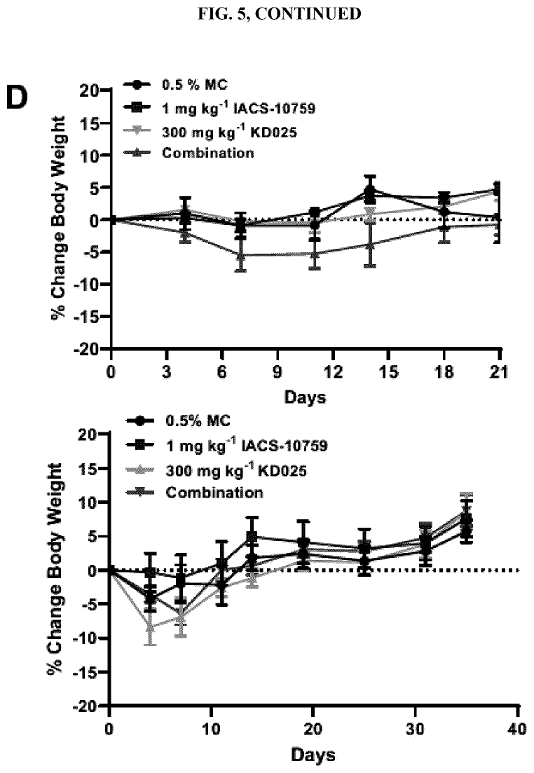

As a single agent, neither inhibitor had a significant effect on tumor growth (FIG. 5, Panel A and Panel C (left panel)). Co-administration of both compounds, however, significantly and synergistically suppressed tumor growth leading to stable disease in the H1299 xenograft model even after 21 days of treatment. These results demonstrate that OXPHOS inhibition, even with low dose IACS-010759, in combination with ROCK inhibition, such as with KD025, is an effective treatment strategy for SMARCA4-mutant cancer patients. Similar results were observed in an additional lung cancer A549 xenograft model (FIG. 5, Panel B, Panel C (right panel)). Importantly, KD025 and IACS-10759 alone or in combination were well tolerated. Mice did not exhibit any significant weight loss or other overt toxicities over the course of the studies (FIG. 5, Panel D). The data support the use of KD025 and low dose IACS-10759 as an effective therapeutic intervention for SMARCA4-mutant lung cancer patients.

### Example 6: Evaluation of Anti-Tumor Activity of the KD025 and IACS-10759 Combination In a Patient Derived Xenograft (PDX) Model In Vivo

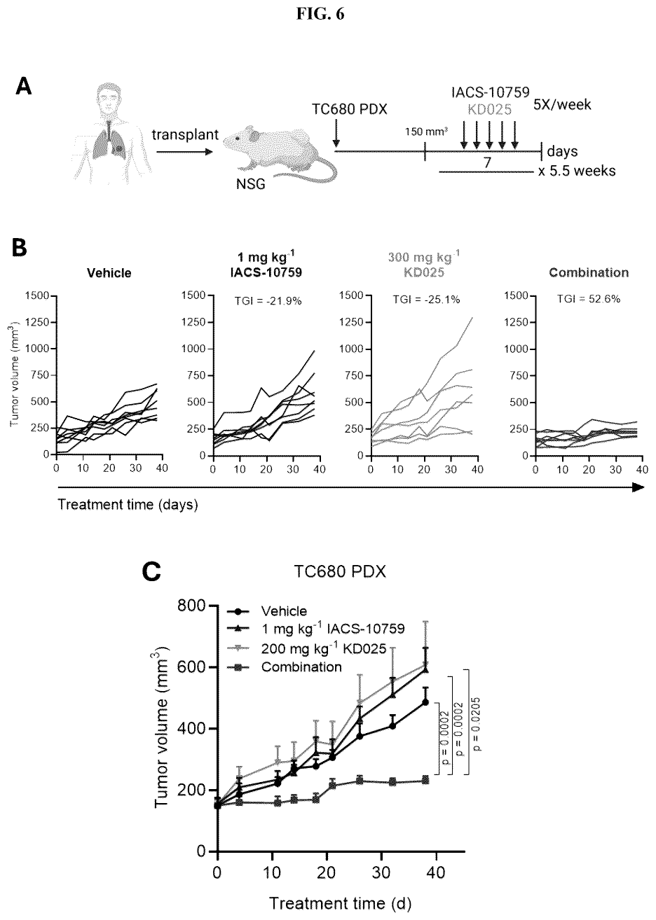

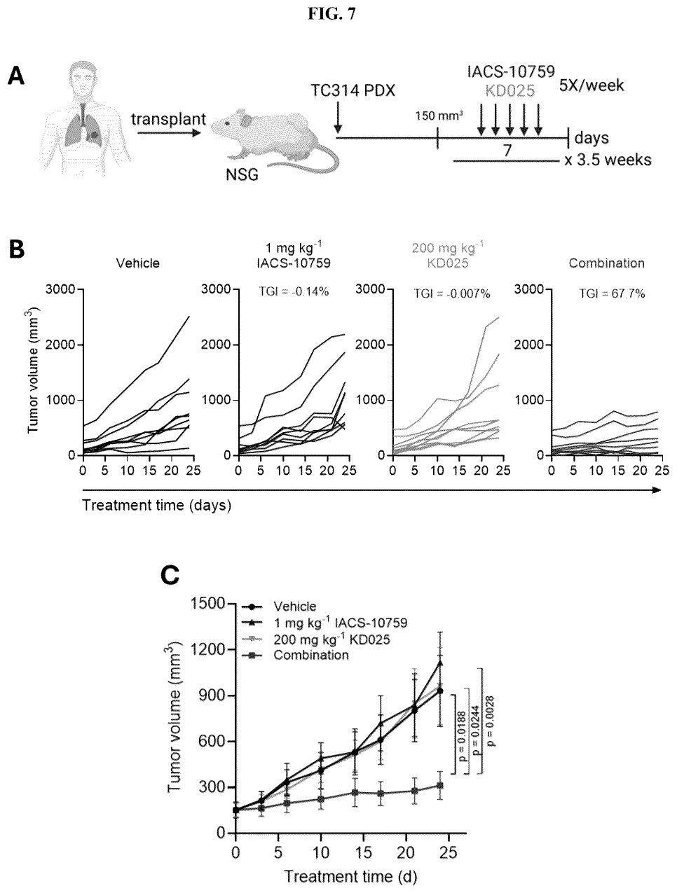

An in vivo study was conducted in a TC680 SMARCA4-mutant PDX model in NSG mice (FIG. 6, Panel A). Briefly, mice were treated with vehicle, 1 mg/kg IACS-10759, 200 mg/kg KD025, or a combination of 1 mg/kg IACS-10759 and 200 mg/kg KD025 by daily oral gavage (5×/week) for 5.5 weeks. As shown in FIG. 6, Panels B and C, the combination of IACS-10759 and KD025 significantly reduced tumor growth compared to vehicle or either treatment alone. The data further support the use of KD025 and low dose IACS-10759 as an effective therapeutic intervention for SMARCA4-mutant lung cancer patients. Similar results were also obtained in a TC314 SMARCA4-mutant PDX model (FIG. 7, Panels A, B, and C).

### Example 7: ROCK Blockade Combined With OXPHOS Inhibition Causes Cell Cycle Growth Arrest and Apoptosis Following Only a Single Dose

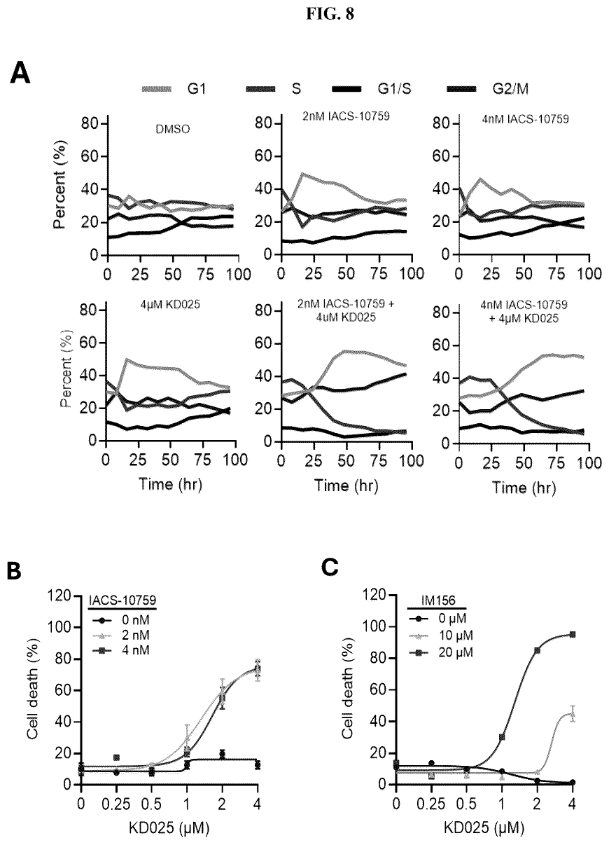

The cell cycle reporter (FUCCI) was used to monitor different phases of the cell cycle in real-time in H1299 cells following single dose administration of IACS-10759 (2 nM or 4 nM), KD025 (4 μM), or a combination of IACS-10759 and KD025 (FIG. 8, Panel A). Cell death was also analyzed following treatment with IACS-10759 and KD025 alone or in combination using (FIG. 8, Panel B) or following treatment with IM156 and KD025 alone or in combination (FIG. 8, Panel C). Profound cell death was observed following combination therapy with IACS-10759 and KD025 or IM156 and KD025.

All of the methods disclosed and claimed herein can be made and executed without undue experimentation in light of the present disclosure. While the compositions and methods of this invention have been described in terms of preferred embodiments or aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of treating a subject afflicted with or at risk of developing a cancer comprising a mutation in a subunit of a SWI/SNF chromatin remodeling complex, the method comprising administering to the subject an effective amount of a ROCK inhibitor and an effective amount of an OXPHOS inhibitor.

2. The method of claim 1, wherein the ROCK inhibitor is selected from the group consisting of belumosudil (KD025), AT-13148, BA-210, β-elemene, chroman 1, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, and Y-39983.

3. The method of claim 1, wherein the OXPHOS inhibitor is selected from the group consisting of IACS-010759 (IACS-10759), metformin, phenformin, HP661, IM156 (lixumistat, HL156A), BAY 87-2243, VLX600, lonidamine, atovaquone, AG311, Mito-Met₁₀ (norMitoMet), mubritinib, carboxyamidotriazole (CAI), ME344, fenofibrate, deguelin, papaverine, α-TOS, neoantimycin F, ADDA 5, Gboxin, S-Gboxin, oligomycin A, apoptolidin, bedaquiline, DX3-213B, BAY-179, 4-methyl-2-oxovaleric acid (ketoleucine, 4-MOV, KIC), diphenylamine hydrochloride, and carbonylcyanide 3-chlorophenylhydrazone (CCCP), carbonyl cyanide 4-(trifluoromethoxy)phenylhydrazone (FCCP), 3-nitropropionic acid, amobarbital, antimycin A, arsenic trioxide, atpenin A5, aurovertin B, BAM 15, Bz-423, berberine, canagliflozin, calcimycin (A-23187), cyanine5 alkyne (alkyne-Cy5), DX2-201, DX3-234, DX3-235, hydrocortisone, IM176OUT05, malonate, mIBG, Mito-LND (Mito-Lonidamine), Mito-Q, MPTP (1-methyl 4-phenyl 1,2,3,6 tetrahydropyridine), myxothiazol nitric oxide, nefazodone, nonactin, parimifasor (LYC-30937), piericidin A, pioglitazone, pyrvinium, ranolazine, rosiglitazone, rotenone, RTB70, TRC1, OXPHOS-IN-1, SMV-32, stigmatellin, TRAP1-IN-2, TRAP1-IN-1, SCAL-255, SCAL-266, siccanin, tetrathiomolybdate, tiabendazole, and TTFA (thenoyltrifluoroacetone).

4. The method of claim 1, wherein the ROCK1 inhibitor is belumosudil (KD025) and the OXPHOS inhibitor is IACS-010759 (IACS-10759), metformin, phenformin, or IM156.

5. The method of claim 1, wherein the subunit of the SWI/SNF chromatin remodeling complex is ARID1A or SMARCA4.

6. The method of claim 1, wherein the cancer is resistant to chemotherapy, immunotherapy, or an inhibitor of KRAS.

7. The method of claim 1, wherein the effective amount of the ROCK1 inhibitor is about 1 mg/kg to about 2500 mg/kg body weight, about 10 mg/kg to about 2000 mg/kg body weight, about 50 mg/kg to about 1750 mg/kg body weight, about 100 mg/kg to about 1500 mg/kg body weight, about 200 mg/kg to about 1200 mg/kg body weight, about 100 mg/kg to about 800 mg/kg body weight, about 100 mg/kg to about 600 mg/kg body weight, about 200 mg/kg boy weight to about 500 mg/kg body weight, or about 200 mg/kg to about 400 mg/kg body weight.

8. The method of claim 7, wherein the effective amount of the ROCK1 inhibitor is about 1 mg/kg to about 2500 mg/kg body weight per day, about 10 mg/kg to about 2000 mg/kg body weight per day, about 50 mg/kg to about 1750 mg/kg body weight per day, about 100 mg/kg to about 1500 mg/kg body weight per day, about 200 mg/kg to about 1200 mg/kg body weight per day, about 100 mg/kg to about 800 mg/kg body weight per day, about 100 mg/kg to about 600 mg/kg body weight per day, about 200 mg/kg boy weight to about 500 mg/kg body weight per day, or about 200 mg/kg to about 400 mg/kg body weight per day.

9. The method of claim 1, wherein the effective amount of the OXPHOS inhibitor is about 0.5 mg/kg to about 2500 mg/kg body weight, about 10 mg/kg to about 2000 mg/kg body weight, about 50 mg/kg to about 1750 mg/kg body weight, about 200 mg/kg to about 1000 mg/kg body weight, about 200 mg/kg to about 800 mg/kg body weight, about 400 mg/kg to about 600 mg/kg body weight, about 0.5 mg/kg to about 20 mg/kg body weight, about 0.5 mg/kg to about 15 mg/kg body weight, about 0.5 mg/kg to about 10 mg/kg body weight, about 0.5 mg/kg to about 9 mg/kg body weight, about 0.5 mg/kg to about 8 mg/kg body weight, about 0.5 mg/kg to about 7 mg/kg body weight, about 0.5 mg/kg to about 6 mg/kg body weight, about 0.5 mg/kg to about 5 mg/kg body weight, about 0.5 mg/kg to about 4 mg/kg body weight, about 0.5 mg/kg to about 3 mg/kg body weight, about 0.5 mg/kg to about 2 mg/kg body weight, or about 0.5 mg/kg to about 1.5 mg/kg body weight.

10. The method of claim 9, wherein the effective amount of the OXPHOS inhibitor is about 0.5 mg/kg to about 2500 mg/kg body weight per day, about 10 mg/kg to about 2000 mg/kg body weight per day, about 50 mg/kg to about 1750 mg/kg body weight per day, about 200 mg/kg to about 1000 mg/kg body weight per day, about 200 mg/kg to about 800 mg/kg body weight per day, about 400 mg/kg to about 600 mg/kg body weight per day, about 0.5 mg/kg to about 20 mg/kg body weight per day, about 0.5 mg/kg to about 15 mg/kg body weight per day, about 0.5 mg/kg to about 10 mg/kg body weight per day, about 0.5 mg/kg to about 9 mg/kg body weight per day, about 0.5 mg/kg to about 8 mg/kg body weight per day, about 0.5 mg/kg to about 7 mg/kg body weight per day, about 0.5 mg/kg to about 6 mg/kg body weight per day, about 0.5 mg/kg to about 5 mg/kg body weight per day, about 0.5 mg/kg to about 4 mg/kg body weight per day, about 0.5 mg/kg to about 3 mg/kg body weight per day, about 0.5 mg/kg to about 2 mg/kg body weight per day, or about 0.5 mg/kg to about 1.5 mg/kg body weight per day.

11. The method of claim 1, wherein the cancer is selected from the group consisting of lung cancer, non-small cell lung cancer (NSCLC), small cell lung cancer (SCLC), brain cancer, glioblastoma, medulloblastoma, skin cancer, melanoma, pancreatic cancer, colorectal cancer, appendiceal cancer, hematopoietic cancer, B-cell lymphoma, leukemia, myeloma, breast cancer, head and neck cancer, prostate cancer, kidney cancer, bladder cancer, liver cancer, esophageal cancer, stomach cancer, thyroid cancer, small bowel adenocarcinoma, hepatobiliary cancer, gynecological cancer, cervical cancer, uterine cancer, and ovarian cancer.

12. The method of claim 1, wherein the subject is a mammalian subject.

13. The method of claim 1, wherein the subject is a human subject.

14. The method of claim 1, wherein said administering comprises oral administration, buccal administration, injection, microneedle administration, vaginal administration, inhalation, intraosseous administration, transnasal application, topical administration, transdermal application, or rectal administration.

15. The method of claim 1, further comprising administering a second therapy to said subject.

16. The method of claim 15, wherein said second therapy is selected from the group consisting of chemotherapy, radiation therapy, immunotherapy, and surgery.

17. The method of claim 1, further comprising administering a pharmaceutical composition comprising the effective amount of the ROCK inhibitor or the effective amount of the OXPHOS inhibitor to said subject.

18. The method of claim 1, further comprising administering a first pharmaceutical composition comprising the effective amount of the ROCK inhibitor and a second pharmaceutical composition comprising the effective amount of the OXPHOS inhibitor to said subject.

19. The method of claim 17, wherein the pharmaceutical composition comprises the effective amount of the ROCK inhibitor and the effective amount of the OXPHOS inhibitor.

20. A pharmaceutical composition comprising an effective amount of a ROCK inhibitor and an effective amount of an OXPHOS inhibitor.

21. The pharmaceutical composition of claim 20, wherein the ROCK inhibitor is selected from the group consisting of belumosudil (KD025), AT-13148, BA-210, β-elemene, chroman 1, DJ4, fasudil, GSK-576371, GSK429286A, H-1152, hydroxyfasudil, ibuprofen, LX-7101, netarsudil, RKI-1447, ripasudil, TCS-7001, thiazovivin, verosudil, Y-27632, Y-30141, Y-33075, and Y-39983.

22. The pharmaceutical composition of claim 20, wherein the OXPHOS inhibitor is selected from the group consisting of IACS-010759 (IACS-10759), metformin, phenformin, HP661, IM156 (lixumistat, HL156A), BAY 87-2243, VLX600, lonidamine, atovaquone, AG311, Mito-Met10 (norMitoMet), mubritinib, carboxyamidotriazole (CAI), ME344, fenofibrate, deguelin, papaverine, α-TOS, neoantimycin F, ADDA 5, Gboxin, S-Gboxin, oligomycin A, apoptolidin, bedaquiline, DX3-213B, BAY-179, 4-methyl-2-oxovaleric acid (ketoleucine, 4-MOV, KIC), diphenylamine hydrochloride, and carbonylcyanide 3-chlorophenylhydrazone (CCCP), carbonyl cyanide 4-(trifluoromethoxy)phenylhydrazone (FCCP), 3-nitropropionic acid, amobarbital, antimycin A, arsenic trioxide, atpenin A5, aurovertin B, BAM 15, Bz-423, berberine, canagliflozin, calcimycin (A-23187), cyanine5 alkyne (alkyne-Cy5), DX2-201, DX3-234, DX3-235, hydrocortisone, IM176OUT05, malonate, mIBG, Mito-LND (Mito-Lonidamine), Mito-Q, MPTP (1-methyl 4-phenyl 1,2,3,6 tetrahydropyridine), myxothiazol nitric oxide, nefazodone, nonactin, parimifasor (LYC-30937), piericidin A, pioglitazone, pyrvinium, ranolazine, rosiglitazone, rotenone, RTB70, TRC1, OXPHOS-IN-1, SMV-32, stigmatellin, TRAP1-IN-2, TRAP1-IN-1, SCAL-255, SCAL-266, siccanin, tetrathiomolybdate, tiabendazole, and TTFA (thenoyltrifluoroacetone).

23. The pharmaceutical composition of claim 20, wherein the ROCK1 inhibitor is belumosudil (KD025) and the OXPHOS inhibitor is IACS-010759 (IACS-10759), metformin, phenformin, or IM156.

24. The pharmaceutical composition of claim 20, wherein said pharmaceutical composition is formulated for oral administration, buccal administration, injection, microneedle administration, vaginal administration, inhalation, intraosseous administration, trans nasal application, topical administration, transdermal application, or rectal administration.

25. The pharmaceutical composition of claim 20, wherein the pharmaceutical composition is serum-free, endotoxin-free, or sterile.
